(19) United States
(12) Patent Application Publication
(10) Pub. No.: US 2025/0206859 A1
(43) Pub. Date: Jun. 26, 2025

(54) LINEAR LOW DENSITY POLYETHYLENES, POLYMERIZATIONS THEREOF, AND FILMS THEREOF

(71) Applicant: ExxonMobil Chemical Patents Inc., Spring, TX (US)

(72) Inventors: Petra EISELT, Houston, TX (US); Hannah K. POHLMANN, Houston, TX (US); Valentin MUNOZ, Baytown, TX (US); Richard E. PEQUENO, Baytown, TX (US); Onur KIR, Brussels (BE); Etienne R.H LERNOUX, Longueville (FR)

(21) Appl. No.: 18/849,968

(22) PCT Filed: Mar. 28, 2023
(86) PCT No.: PCT/US2023/065021

§ 371 Date: Sep. 23, 2024

Related U.S. Application Data
(60) Provisional application No. 63/362,239, filed on Mar. 31, 2022.

(51) Int. Cl. C08F 210/16; C08J 5/18; G01N 33/44

(52) U.S. Cl. CPC C08F 210/16; C08J 5/18; G01N 33/442; C08F 2800/20; C08J 2323/08

(57) ABSTRACT

This disclosure relates to polyethylene polymers, polymerization processes for making such polyethylene polymers, and films made therefrom. In some embodiments, a polyethylene copolymer includes ethylene units; and 1 wt % to 8 wt % of Cs-Cs alpha-olefin comonomer units. The polyethylene copolymer has a density of 0.914 g/cm3 to 0.925 g/cm3 and a melt index (MI, determined per ASTM D1238 at 190° C. and 2.16 kg loading) greater than 1 g/10 min and less than or equal to 2.5 g/10 min. The polyethylene copolymer has a composition distribution breadth index of 75% or greater and a molecular weight distribution (M_{w}/Mₙ) of 2 to 8.

### CROSS-REFERENCE TO RELATED APPLICATION

This application claims the benefit of U.S. Provisional Application 63/362,239, filed Mar. 31, 2022, entitled “LINEAR LOW DENSITY POLYETHYLENES, POLYMERIZATIONS THEREOF, AND FILMS THEREOF”, the entirety of which is incorporated by reference herein.

### FIELD

This disclosure relates to polyethylene polymers, polymerization processes for making such polyethylene polymers, and films made therefrom.

### BACKGROUND

A linear low density polyethylene (LLDPE) is a substantially linear polymer composed of ethylene monomeric units and alpha-olefin comonomeric units. The typical comonomeric units used are derived from 1-butene, 1-hexene, or 1-octene. An LLDPE may be distinguished from a conventional low density polyethylene (LDPE) in several ways including their different manufacturing processes. In addition, LLDPE has little or no detectable long chain branching (LCB) per 1,000 carbon atoms, whereas conventional LDPEs contain long chain branching. Long chain branching provides reduced neck-in and increased draw stability during extrusion processes. In addition, LLDPEs have a narrower molecular weight distribution (MWD) relative to MWD of LDPEs. LLDPEs also have different rheological and mechanical properties, such as tear properties, as compared to LDPEs.

An LLDPE formed using a metallocene catalyst is known as an “mLLDPE”. Extrusions of mLLDPEs need more motor power and higher extruder pressures to match the extrusion rates of LDPEs. Typical mLLDPEs also have lower melt strength which, for example, adversely affects bubble stability during blown film extrusion, and mLLDPEs are prone to melt fracture at commercial shear rates. Melt fracture is flow disturbance leading to surface roughness and/or surface irregularities in the extruded resin involving a severe distortion of the extrudate. Melt fracture occurs when shear stress imparted into the resin exceeds the critical shear stress value of that resin or slip-stick flow conditions occur in the die. Melt fracture can be related to high die shear rates (e.g., 1,000-60,000 s⁻¹) and shear stresses of such mLLDPEs used to form films. The high shear rates are a result of high line speeds (e.g., >600 m/min) used to achieve thin films of the mLLDPE.

For many polyolefin applications, including films and fibers, increased melt strength is a desirable attribute. Higher melt strength allows fabricators to run blown film lines at a faster rate to form films of the mLLDPE. To ameliorate and compensate for the above-mentioned melt fracture during extrusion, fluorinated additives, such as PFAS (per- or poly-fluoroalkanes), are typically mixed with the mLLDPE. However, such fluorinated additives are increasingly receiving regulatory scrutiny.

Regardless of the above processing and rheological challenges, mLLDPEs do exhibit superior physical properties as compared to LDPEs. In the past, various levels of LDPE have been blended with mLLDPEs to increase melt strength, to increase shear sensitivity, e.g. to increase flow at commercial shear rates in extruders, and to reduce the tendency to melt fracture. However, such blends generally have poor mechanical properties as compared with neat mLLDPEs. Indeed, it has been a challenge to improve mLLDPEs processability without sacrificing physical properties.

Comparing LLDPEs to one another, an LLDPE having a higher melt index is better for processing, and a combination of higher melt index and lower density is particularly good for cast film applications. However, less long chain branching can lead to reduced film properties. Indeed, it is a challenge to find an LLDPE having a combination of density and melt index while still being commercially processable.

In addition to processing challenges described above, polymerizations to form mLLDPEs also present their own challenges. For example, a polyethylene having a higher melt index and lower density is expected to have a sticky consistency such that, when formed in a reactor, the polyethylene will stick to the walls and other components of the reactor, leading to a reactor shutdown.

Some references of potential interest in this regard include: U.S. Pat. Nos. 6,255,426; 7,951,873; 9,718,896; and 10,029,226; as well as US Patent Publication Nos. US2004/0121098; US20070260016; US2015/0232589; ad US2020/0339715.

Overall, there is a need for new LLDPEs having a combination of desirable properties (such as density, melt index properties, long chain branching) while also providing commercially desirable polymerizations and extrusions of the LLDPEs.

### BRIEF SUMMARY

In some embodiments, a polyethylene copolymer includes ethylene units; and 1 wt % to 8 wt % of C₃-C₈ alpha-olefin comonomer units. The polyethylene copolymer has a density of 0.914 g/cm³ to 0.925 g/cm³, a melt index of 1 g/10 min to 2.5 g/10 min, composition distribution broadness described as T₇₅−T₂₅ (° C.) of 5.8-10, a molecular weight distribution of 2 to 8, and a melt index ratio (MIR, defined as ratio of high load melt index (HLMI) to melt index (MI)) within the range from 25 to 35.

In some embodiments, a polyethylene copolymer includes ethylene units; and 1 wt % to 8 wt % of C₃-C₈ alpha-olefin comonomer units. The polyethylene copolymer has a density of 0.914 g/cm³ to 0.925 g/cm³ and a melt index (MI, determined per ASTM D1238 at 190° C. and 2.16 kg loading) greater than 1 g/10 min and less than or equal to 2.5 g/10 min. The polyethylene copolymer has a composition distribution breadth index of 75% or greater and a molecular weight distribution (M_{w}/Mₙ) of 2 to 8.

In some embodiments, a cast film includes a polyethylene copolymer. The polyethylene copolymer has a density of 0.915 g/cm³ to 0.917 g/cm³ and a melt index of 1.6 g/10 min to 1.8 g/10 min.

In some embodiments, a blown film includes a polyethylene copolymer. The polyethylene copolymer has a density of 0.915 g/cm³ to 0.922 g/cm³ and a melt index of 1.5 g/10 min to 2.3 g/10 min.

In some embodiments, a method of modeling a stickiness temperature for a polyethylene copolymer is included, wherein the polyethylene copolymer is made in the presence of at least one catalyst. The method includes measuring a stickiness temperature of the polyethylene copolymer at each of a plurality of concentrations of an induced condensing agent in a testing device. The method includes measuring a density, a melt index (MI), and a high load melt index (HLMI) of the polyethylene copolymer. The method includes calculating a melt flow ratio by dividing the HLMI by the MI. The method includes calculating an equivalent partial pressure of the induced condensing agent by accounting for the partial pressure of isomers that accumulate in a reactor. The method includes determining an equation that relates the stickiness temperature to the equivalent partial pressure, based, at least in part, on the density, the MI, and the MFR of the polyethylene copolymer. The polyethylene copolymer includes ethylene units and 1 wt % to 8 wt % of C₃-C₈ alpha-olefin comonomer units. The polyethylene copolymer has a density of 0.914 g/cm³ to 0.925 g/cm³, a melt index of 1 g/10 min to 2.5 g/10 min, a composition distribution broadness characterized by T₇₅−T₂₅ (° C.) of 5.8-10, a molecular weight distribution of 2 to 8, and a melt index ratio (MIR, defined as HLMI/MI) within the range from 25 to 35. The method can include using the modeled stickiness temperature (e.g., using the equation relating the stickiness temperature to the equivalent partial pressure) to produce additional polyethylene copolymer (e.g., in a polymerization reactor, said polymerization taking place in the presence of a polymerization catalyst).

In some embodiments, a method of controlling a polymerization to remain in a non-sticking regime is provided. The method includes measuring parameters for the polymerization reaction including a reactor temperature and a concentration of an induced condensing agent (ICA) in a polymerization reactor. The method includes calculating an equivalent partial pressure of the ICA. The method includes locating the polymerization reaction in a two dimension space defined by a reactor temperature dimension and an equivalent partial pressure dimension. The method includes comparing the location in the two dimensional space to a non-sticking regime defined as a space between an upper temperature limit curve and a lower temperature limit curve. The method includes adjusting one or more parameters of the polymerization to maintain the polymerization within the non-sticking regime. The method includes obtaining a polyethylene copolymer from the polymerization reactor. The polyethylene copolymer includes ethylene units and 1 wt % to 8 wt % of C₃-C₈ alpha-olefin comonomer units. The polyethylene copolymer has a density of 0.914 g/cm³ to 0.925 g/cm³, a melt index of 1 g/10 min to 2.5 g/10 min, a composition distribution breadth index of 75% or greater, a molecular weight distribution of 2 to 8, and a melt index ratio (HLMI/MI) within the range from 25 to 35.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a process flow diagram of a method for measuring stickiness temperature, according to an embodiment.

FIG. 2 is a process flow diagram of a method of operating a reactor in a non-sticking regime, according to an embodiment.

FIG. 3 is a plot of melt index versus density of conventional and inventive resins, according to an embodiment.

FIG. 4A is a gel permeation chromatography chromatogram of inventive and comparative resins, according to an embodiment.

FIG. 4B is a graph illustrating density versus EEE triad content of inventive and comparative resins, according to an embodiment.

FIG. 5 is a graph illustrating viscosity flow curves at 190° C. of inventive and comparative resins, according to an embodiment.

FIG. 6 is a graph illustrating VGP (Van Gurp Palmen) curves for inventive and comparative resins, according to an embodiment.

FIG. 7 is a graph illustrating temperature rising elution fractionation (TREF) chromatograms of inventive and comparative resins, according to an embodiment.

FIG. 8 is a graph illustrating melt strength of an inventive resin, according to an embodiment.

FIG. 9 is a plot illustrating motor load, melt pressure, and melt temperature of film formation using an inventive resin and a comparative resin, according to an embodiment.

FIG. 10 is a plot illustrating motor load, melt pressure, and melt temperature of film formation using an inventive resin and a comparative resin, according to an embodiment.

### DETAILED DESCRIPTION

Various embodiments, versions of the disclosed compounds, processes, and articles of manufacture, will now be described, including specific embodiments and definitions that are adopted herein. While the following detailed description gives specific embodiments, those skilled in the art should appreciate that these embodiments are exemplary only, and that embodiments of the present disclosure can be practiced in other ways. Any reference to embodiments may refer to one or more, but not necessarily all, of the compounds, processes, or articles of manufacture defined by the claims. The use of headings is for purposes of convenience only and does not limit the scope of the present disclosure.

This disclosure relates to polyethylene polymers, polymerization processes for making such polyethylene polymers, and films made therefrom. Polyethylene polymers are copolymers having a combination of low density, high melt index, maintained long chain branching, and relatively consistent comonomer incorporation across polymer chains of different lengths while also providing commercially desirable polymerizations and extrusions of the polyethylene copolymers.

As compared to conventional LLDPEs, polyethylene copolymers of the present disclosure can maintain a small amount of long chain branching (also referred to as “LCB”) in the copolymers providing reduced neck-in and increased draw stability. Polyethylene copolymers of the present disclosure can exhibit lower zero shear viscosity, leading to lower motor torque and lower melt pressures and melt temperatures during extrusion, providing increased output of the extruded polyethylene copolymer product. In addition, because LCB is maintained, advantageous processing properties are likewise maintained. For example, an ˜10% reduction in motor torque and ˜25% reduction in melt pressure may be observed during cast film fabrication (e.g., 13 of FIG. 8). This small amount of LCB can be evidenced through, e.g., a high melt index ratio and/or particular rheology characteristics as shown through data obtained by small angle oscillatory shear (SAOS) experiments (for instance, ratio of η_{0.01}/η₁₀₀, the complex viscosity recorded at shear rates of 0.01 and 100 rad/s, respectively).

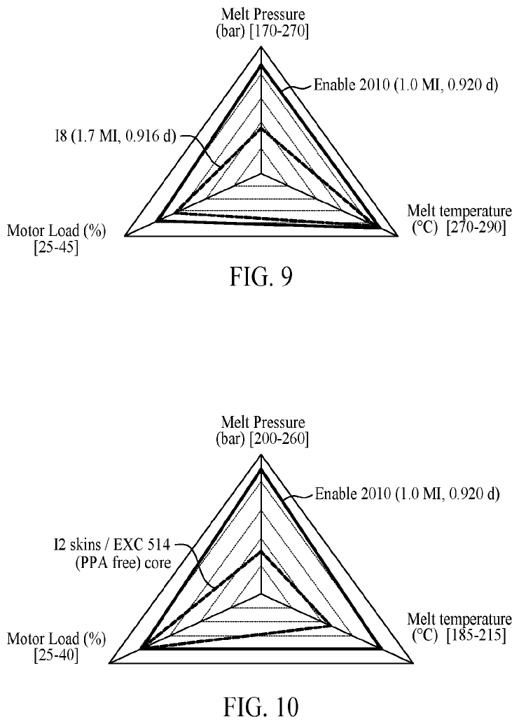

In addition, it has been discovered that polyethylene copolymers of the present disclosure can provide excellent shear thinning characteristics with little or no melt fracture of the extrudate at high die shear rates. For example, polyethylene copolymers of the present disclosure having small amount of LCB in combination with an MI of 1.5 to 2.5 g/10 min and a density of about 0.914 to 0.925 g/cm³ can provide blown films having excellent bubble stability (little or no melt fracture) which allows polymer processing aids (such as per- or poly-fluoro alkanes) to be merely optional in blown films of the present disclosure. Polyethylene copolymers of the present disclosure (e.g., small amount of LCB in combination with MI of 1.5 to 2.5 and a density of 0.914 to 0.925 g/cm³) further can provide films formed with reduced motor load and melt pressure (which increases throughput) due to improved flow behavior, as compared to conventional LLDPEs. For example, an ˜18% reduction in melt pressure and an ˜12° C. decrease in melt temperature may be provided during blown film fabrication (e.g., see FIG. 9).

### Definitions

As used herein, an “olefin,” alternatively referred to as “alkene,” is a linear, branched, or cyclic compound of carbon and hydrogen having at least one double bond. For purposes of this specification and the claims appended thereto, when a polymer or copolymer is referred to as “comprising” an olefin, the olefin present in such polymer or copolymer is the polymerized form of the olefin. For example, when a copolymer is described as having an “ethylene” content of 35 wt % to 55 wt %, it is understood that the mer unit in the copolymer is derived from ethylene in the polymerization reaction and the derived units are present at 35 wt % to 55 wt %, based upon the weight of the copolymer.

As used herein, the terms “polyethylene polymer,” “polyethylene,” “ethylene polymer,” “ethylene copolymer,” and “ethylene based polymer” mean a polymer or copolymer comprising at least 50 mol % ethylene units, or at least 70 mol % ethylene units, or at least 80 mol % ethylene units, or at least 90 mol % ethylene units, or at least 95 mol % ethylene units or 100 mol % ethylene units (in the case of a homopolymer).

As used herein, a “polymer” may refer to homopolymers, copolymers, interpolymers, terpolymers, etc. A “polymer” has two or more of the same or different monomer units. A “homopolymer” is a polymer having monomer units that are the same. A “copolymer” is a polymer having two or more monomer units that are different from each other. A “terpolymer” is a polymer having three monomer units that are different from each other. The term “different” as used to refer to monomer units indicates that the monomer units differ from each other by at least one atom or are different isomerically. Accordingly, the definition of copolymer, as used herein, includes terpolymers and the like. Likewise, the definition of polymer, as used herein, includes copolymers and the like.

As used herein, an ethylene polymer having a density of more than 0.860 to less than 0.910 g/cm³ is referred to as an ethylene plastomer or plastomer; an ethylene polymer having a density of 0.910 to 0.925 g/cm³ is referred to as a “linear low density polyethylene” (LLDPE) when substantially linear (having minor or no long chain branching) as is typically the case for Ziegler-Nata or metallocene-catalyzed PE or branched low density polyethylene (LDPE) when significantly branched (having a high degree of long chain branching), as is often the case with free-radical polymerized PE; 0.925 to 0.940 g/cm³ is referred to as a “medium density polyethylene” (MDPE); and an ethylene polymer having a density of more than 0.940 g/cm³ is referred to as a “high density polyethylene” (HDPE). Density is determined according to ASTM D792. Specimens are prepared according to ASTM D4703—Annex 1 Procedure C followed by conditioning according to ASTM D618—Procedure A prior to testing.

As used herein, and unless otherwise specified, the term “Cn” means hydrocarbon(s) having n carbon atom(s) per molecule, wherein n is a positive integer.

As used herein, and unless otherwise specified, the term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated or unsaturated), including mixtures of hydrocarbon compounds having different values of n.

As used herein, the term “film” refers to a continuous, flat (in some instances, flexible) polymeric structure having an average thickness of a range of 0.1, or 1, or 5, or 10, or 15, or 20 μm to50, or 75, or 100, or 150, or 200, or 250, or 1000, or 2000 μm, or such a coating of similar thickness adhered to a flexible, non-flexible or otherwise solid structure. The “film” may be made from or contain a single layer or multiple layers. Each layer may be made from or contain the polyethylene copolymers of the present disclosure. For example, one or more layers of a “film” may include a mixture of the disclosed polyethylene copolymer as well as a LDPE, another LLDPE, polypropylene, or a plastomer.

As used herein, a composition or film “free of” a component refers to a composition/film substantially devoid of the component, or comprising the component in an amount of less than about 0.01 wt %, by weight of the total composition.

As used herein, the term “polymerizable conditions” refers to conditions conducive to the reaction of one or more olefin monomers when contacted with an activated olefin polymerization catalyst to produce a polyolefin polymer, including a skilled artisan's selection of temperature, pressure, reactant concentrations, optional solvent/diluents, reactant mixing/addition parameters, and other conditions within at least one polymerization reactor.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, “in a range” or “within a range” includes every point or individual value between its end points even though not explicitly recited and includes the end points themselves. Thus, every point or individual value may serve as its own lower or upper limit combined with any other point or individual value or any other lower or upper limit, to recite a range not explicitly recited.

The term “terminal olefin” refers to an olefin having a terminal carbon-to-carbon double bond in the structure thereof ((R¹R²)—C═CH₂, where R¹ and R² can be independently hydrogen or any hydrocarbyl group, such as R¹ is hydrogen, and R² is an alkyl group). A “linear terminal olefin” is a terminal olefin defined in this paragraph wherein R¹ is hydrogen, and R² is hydrogen or a linear alkyl group.

The term “vinyl” means an olefin having the following formula:

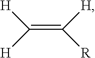

wherein R is a hydrocarbyl group, such as a saturated hydrocarbyl group such as an alkyl group.

The term “vinylidene” means an olefin having the following formula:

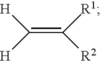

wherein R¹ and R² are each, independently, a hydrocarbyl group, such as a saturated hydrocarbyl group such as alkyl group.

The term “vinylene” or “1,2-di-substituted vinylene” means
(i) an olefin having the following formula:

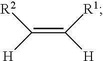

(ii) an olefin having the following formula:

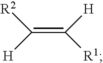

(iii) a mixture of (i) and (ii) at any proportion thereof,
wherein R¹ and R² are each, independently, a hydrocarbyl group, such as saturated hydrocarbyl group such as alkyl group.

The term “tri-substituted vinylene” means an olefin having the following formula:

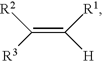

wherein R¹, R², and R³ are each, independently, a hydrocarbyl group, such as a saturated hydrocarbyl group such as alkyl group.

### Polyethylene Copolymers

The present disclosure provides polyethylene copolymers having a combination of low density, high melt index, maintained small amount of long chain branching, and relatively consistent comonomer incorporation across polymer chains of different lengths (high composition distribution breadth index (CDBI)). In addition, the polyethylene copolymers and films thereof can be formed by commercially desirable polymerizations and extrusions of the polyethylene copolymers.

Thus, polyethylene copolymers of various embodiments herein can exhibit one or more of the following properties:
Density within the range from 0.914 to 0.925 g/cm³, such as from a low of any one of 0.914, 0.915, 0.916, 0.917, 0.918, 0.919, or 0.92 g/cm3 to a high of any one of 0.925, 0.924, 0.923, 0.922, 0.921, 0.920, or 0.919 g/cm³, such as 0.915 g/cm³ to 0.920 g/cm³, alternatively 0.918 g/cm³ to 0.922 g/cm³, with combinations from any low to any high contemplated (provided the high end is greater than the low end), e.g., 0.916 to 0.921 g/cm³.
Melt Index (MI, also referred to as I₂ or I_{2.16} in recognition of the 2.16 kg loading used in the test) greater than 1.0 g/10 min (ASTM D1238, 190° C., 2.16 kg), such as within the range from a low of any one of 1, 1.1, 1.2, 1.3, 1.4, 1.5, 1.6, 1.7, 1.8, 1.9, 2, 2.1, 2.2, 2.3, 2.4, or 2.5 g/10 min to a high end of any one of 1.8, 1.9, 2.0, 2.1, 2.2, 2.3, 2.4, 2.5, 2.6, 2.7, 2.8, 2.9, 3, 4, or 5 g/10 min, with ranges from any low end to any high end contemplated herein (provided the high end is greater than the low end), such as 1 to 2.5 g/10 min, or 1.5 to 2 g/10 min, such as 1.7 g/10 min, etc.
Comonomer distribution reflecting a similar degree of comonomer incorporation on polymer chains of varying length of the polyethylene copolymer, which is quantified in the composition distribution breadth index (CDBI). For instance, polyethylene comonomers of various embodiments have CDBI of 70% or more, such as 75% or more, such as 80% or more, 85% or more, or even 90% or more. CDBI is defined as the weight percent of the copolymer molecules having a comonomer content within 50% of the median total molar comonomer content (i.e., within a range from 0.5× median to 1.5× median), and it is referenced, e.g., in U.S. Pat. No. 5,382,630. In general, copolymers with a broader distribution result in a lower CDBI, while a theoretical copolymer with exactly the same relative comonomer content across all different lengths of polymer chains would have a CDBI of 100%. The CDBI of a copolymer is readily determined utilizing a technique for isolating individual fractions of a sample of the copolymer. One such technique is generation of a solubility distribution curve using Temperature Rising Elution Fraction (TREF), as described in WO 1993003093 (which in turn references Wild, et al., J. Poly. Sci., Poly. Phys. Ed., vol. 20, p. 441 (1982) and U.S. Pat. No. 5,008,204 in this regard). All three of the foregoing publications are incorporated herein by reference. Alternatively, the narrow comonomer distribution can be reflected in the T₇₅−T₂₅ value, wherein T₂₅ is the temperature at which 25% of the eluted polymer is obtained and T₇₅ is the temperature at which 75% of the eluted polymer is obtained, both in a TREF experiment as described in US2019/0119413 (especially in paragraphs [0055]-[0058]thereof, which description is incorporated by reference herein). A narrow distribution is reflected in the relatively small difference in the T₇₅−T₂₅ value being less than 10° C., such as within the range from 4 to 10° C., such as from a low of any one of 4, 4.5, 5, 5.5, or 6° C. to a high of any one of 10, 9.5, 9, 8.5, 8, 7.5, 7, or 6.5° C., such as 5.8° C. to 7° C., alternatively 7° C. to 9° C., with combinations from any low to any high contemplated (provided the high end is greater than the low end).

The polyethylene copolymer may be the polymerization product of an ethylene monomer and one or more olefin comonomers, such as alpha-olefin comonomers. Alpha-olefin comonomers can have 3 to 12 carbon atoms, or from 4 to 10 carbon atoms, or from 4 to 8 carbon atoms. Olefin comonomers can be selected from the group consisting of propylene, 1-butene, 1-pentene, 1-hexene, 1-heptene, 1-octene, 4-methylpent-1-ene, 1-nonene, 1-decene, 1-undecene, 1-dodecene, 1-hexadecene, and the like, and any combination thereof, such as 1-butene, 1-hexene, and/or 1-octene. In some embodiments, a polyene is used as a comonomer. In some embodiments, the polyene is selected from the group consisting of 1,3-hexadiene, 1,4-hexadiene, cyclopentadiene, dicyclopentadiene, 4-vinylcyclohex-1-ene, methyloctadiene, 1-methyl-1,6-octadiene, 7-methyl-1,6-octadiene, 1,5-cyclooctadiene, norbornadiene, ethylidene norbomene, 5-vinylidene-2-norbomene, 5-vinyl-2-norbomene, and olefins formed in situ in the polymerization medium. In some embodiments, comonomers are selected from the group consisting of isoprene, styrene, butadiene, isobutylene, chloroprene, acrylonitrile, and cyclic olefins. In some embodiments, combinations of the olefin comonomers are utilized. In some embodiments, the olefin comonomer is selected from the group consisting of 1-butene and 1-hexene. The olefin comonomer content of the polyethylene copolymer can range from a low of about 0.1, 5, 5.5, 6, 6.5, 7, 7.5, 8, or 8.5 wt % to a high of about 20, 15, 13, 12.5, 12, 11.5, 11, 10.5, 10, 9.5, or 9 wt %, on the basis of total weight of monomers in the polyethylene copolymer. The balance of the polyethylene comonomer is made up of units derived from ethylene (e.g., from a low of 80, 85, 88, 90, 91, 92, 92.5, 93, 93.5, or 94 wt % to a high of 90, 91, 92, 92.5, 93, 93.5, 94, 94.5, 95, 95.5, 96, 97, 99, or 99.9 wt %). Ranges from any foregoing low end to any foregoing high end are contemplated herein (e.g., 88 to 93 wt %, such as 90 to 92.0 wt % ethylene-derived units and the balance olefin comonomer-derived content).

The polyethylene copolymers can also have a high load melt index (HLMI) (also referred to as I₂₁ or I_{21.6} in recognition of the 21.6 kg loading used in the test) within the range from a low of 40, 45, 50, or 55 g/10 min to a high of 75, 70, 65, 60 or 55 g/10 min; with ranges from any of the foregoing lows to any of the foregoing highs contemplated herein (e.g., 45 to 70 g/10 min, such as 45 to 55 g/10 min, alternatively 60 to 70 g/10 min). The term “high load melt index” (“HLMI”), is the number of grams extruded in 10 minutes under the action of a standard load (21.6 kg) and is an inverse measure of viscosity. As provided herein, HLMI (I₂₁) is determined according to ASTM D1238 (190° C./21.6 kg) and is also sometimes referred to as I₂₁ or I_{21.6}.

The polyethylene copolymers can also have a melt index ratio (MIR, defined as the ratio of 1216/12.16) within the range from a low of any one of 20, 25, 26, 27, 28, 29, 30, or 31 to a high of any one of 40, 35, 34, 33, 32, 31, or 30 with ranges from any of the foregoing lows to any of the foregoing highs contemplated herein (e.g., 27 to 33, such as 28 to 32, or 29 to 31)MIR is the ratio of 121/12.

The polyethylene copolymers can also have a molecular weight distribution (MWD) of about 2 to about 8. The MWD can also range from a low of about 2, 2.5, 3, 3.1, 3.2, 3.3, 3.4, 3.5, 3.6, 3.7, 3.8, 3.9, or 4 to a high of about 3.5, 3.6, 3.7, 3.8, 3.9, 4, 4.1, 4.2, 4.3, 4.4, 4.5, 5, 6, or 8, with ranges from any foregoing low to any foregoing high contemplated, provided the high end of the range is greater than the low end. MWD is defined as the weight average molecular weight (Mw) divided by number-average molecular weight (Mn).

Weight-average molecular weight (Mw) of polyethylene copolymers of various embodiments may be within the range from 70,000 to 95,000 g/mol, such as 75,000 to 90,000 g/mol, such as 75,000 to 85,000 g/mol, alternatively 80,000 to 90,000 g/mol, with ranges from any foregoing low end to any foregoing high end contemplated.

Number-average molecular weight (Mn) of polyethylene copolymers of various embodiments may be within the range from 20,000 to 40,000 g/mol, such as 20,000 to 30,000 g/mol, such as 20,000 to 25,000 g/mol, with ranges from any foregoing low end to any foregoing high end contemplated.

Z-average molecular weight (Mz) of polyethylene copolymers of various embodiments may be within the range from 150,000 to 190,000 g/mol, such as 154,000 to 175,000 g/mol, or 160,000 to 185,000 g/mol, such as 170,000 to 180,000 g/mol, with ranges from any foregoing low end to any foregoing high end contemplated (e.g., 150,000 to 175,000 g/mol or 154,000 to 180,000 g/mol).

Polyethylene copolymers of various embodiments may also exhibit a small (but non-zero) amount of long-chain branching. As noted previously, this may be evidenced through, e.g., SAOS viscosity data (especially η_{0.01}/η₁₀₀) and/or MIR. Further, LCB index (g′ or alternatively g′vis) could be less than 1, such as within the range from 0.9 to 0.99 or 0.94 to 0.98, although still substantially higher than g′ for heavily-LCB polyethylene, such as LDPE made using free radical polymerization. Another useful parameter for indicating the presence of LCB is illustrated in FIG. 6 (discussed in more detail in connection with the Examples, below): Van Gurp Palmen (VGP) plots. In particular, polyethylene copolymers (even LLDPE) with some LCB will exhibit an inflection point in their VGP curve, while LLDPE without any LCB present (e.g., the XP8318 of FIG. 6) show no such inflection point.

The distributions and the moments of molecular weight (Mw, Mn, Mw/Mn, etc.), and the branching index (g′vis) are determined by using a high temperature Gel Permeation Chromatography (Polymer Char GPC-IR) equipped with a multiple-channel band-filter based Infrared detector IR5, an 18-angle Wyatt Dawn Heleos light scattering detector and a 4-capillary viscometer with Wheatstone bridge configuration. Three Agilent PLgel 10-μm Mixed-B LS columns are used to provide polymer separation. Aldrich reagent grade 1,2,4-trichlorobenzene (TCB) with 300 ppm antioxidant butylated hydroxytoluene (BHT) is used as the mobile phase. The TCB mixture is filtered through a 0.1-μm Teflon filter and degassed with an online degasser before entering the GPC instrument. The nominal flow rate is 1.0 ml/min and the nominal injection volume is 200 μL. The whole system including transfer lines, columns, and viscometer detector are contained in ovens maintained at 145° C. The polymer sample is weighed and sealed in a standard vial with 80-μL flow marker (Heptane) added to it. After loading the vial in the autosampler, polymer is automatically dissolved in the instrument with 8 ml added TCB solvent. The polymer is dissolved at 160° C. with continuous shaking for about 2 hour. The concentration (c), at each point in the chromatogram is calculated from the baseline-subtracted IR5 broadband signal intensity (I), using the following equation: c=βI, where β is the mass constant. The mass recovery is calculated from the ratio of the integrated area of the concentration chromatography over elution volume and the injection mass which is equal to the pre-determined concentration multiplied by injection loop volume. The conventional molecular weight (IR M_{w}) is determined by combining universal calibration relationship with the column calibration which is performed with a series of monodispersed polystyrene (PS) standards ranging from 700 to 10 million g/mol. The M_{w} at each elution volume is calculated with the following equation:

$$log M = \frac{log ( {K}_{P S} / K )}{a + 1} + \frac{{a}_{P S} + 1}{a + 1} log {M}_{P S}$$

where the variables with subscript “PS” stand for polystyrene while those without a subscript are the test samples. In this method, α_{PS}=0.67 and K_{PS}=0.000175 while a and K are for ethylene-hexene copolymers as calculated from empirical equations (Sun, T. et al. Macromolecules 2001, 34, 6812), in which a=0.695 and K=0.000579(1-0.75 Wt), where Wt is the weight fraction for hexane comonomer. It should be noted that the comonomer composition is determined by the ratio of the IR5 detector intensity corresponding to CH2 and CH3 channel calibrated with a series of PE and ethylene-hexene homo/copolymer standards whose nominal values are predetermined by NMR or FTIR. Here the concentrations are expressed in g/cm³, molecular weight is expressed in g/mol, and intrinsic viscosity (hence K in the Mark-Houwink equation) is expressed in dL/g.

The LS molecular weight (M) at each point in the chromatogram is determined by analyzing the LS output using the Zimm model for static light scattering

$$\frac{{K}_{o} c}{Δ R ( θ )} = \frac{1}{M P ( θ )} + 2 {A}_{2} c .$$

Here, ΔR(θ) is the measured excess Rayleigh scattering intensity at scattering angle θ, c is the polymer concentration determined from the IR5 analysis, A₂ is the second virial coefficient, P(θ) is the form factor for a monodisperse random coil, and Ko is the optical constant for the system:

$${K}_{o} = \frac{4 {π}^{2} {{n}^{2} ( dn / d c )}^{2}}{{λ}^{4} {N}_{A}}$$

where N_{A} is Avogadro's number, and (dn/dc) is the refractive index increment for the system. The refractive index, n=1.500 for TCB at 145° C. and λ=665 nm. For purposes of the present disclosure and the claims thereto (dn/dc)=0.1048 for ethylene-hexene copolymers.

A high temperature Polymer Char viscometer, which has four capillaries arranged in a Wheatstone bridge configuration with two pressure transducers, is used to determine specific viscosity. One transducer measures the total pressure drop across the detector, and the other, positioned between the two sides of the bridge, measures a differential pressure. The specific viscosity, ηₛ, for the solution flowing through the viscometer is calculated from their outputs. The intrinsic viscosity, [η], at each point in the chromatogram is calculated from the equation [η]=ηₛ/c, where c is concentration and is determined from the IR5 broadband channel output.

The branching index (g′ᵥᵢₛ) is calculated using the output of the GPC-IR5-LS-VIS method as follows. The average intrinsic viscosity, [η]_{avg}, of the sample is calculated by:

$${[ η ]}_{a v g} = \frac{\sum {{c}_{1} [ η ]}_{1}}{\sum {c}_{1}}$$

where the summations are over the chromatographic slices, i, between the integration limits. The branching index g′ᵥᵢₛ is defined as

$${g}_{vis}^{′} = \frac{{[ η ]}_{avg}}{K {M}_{v}^{α}} ,$$

where M_{V} is the viscosity-average molecular weight based on molecular weights determined by LS analysis and the K and α are for the reference linear polymer, which are, for purposes of the present disclosure, α and K are the same as described above for linear polyethylene polymers.

Furthermore, the polyethylene copolymers can have a complex shear viscosity (η*) @0.01 rad/sec and 190° C. in the range of 5,000 to 12,000 Pa·s; or from a low of any one of 5,000; 6,000; 7,000; 8,000; 9,000; 10,000; or 11,000 Pa·s, to a high of any one of 12,000; 11,000; 10,000; 9,000; 8,000; 7,000; or 6,000 Pa·s, with ranges from any low end to any high end contemplated (e.g., 6,000 to 8,000 Pa·s).

Complex shear viscosity (η*)@100 rad/sec and 190° C. may be in the range from 900 to 2000 Pa·s; such as from a low end of any one of 900; 1,000; 1,100; or 1,200 Pa·s to a high end of any one of 1,200; 1,300; 1,400; 1,500; or 2,000 Pa·s, with ranges from any foregoing low to any foregoing high also contemplated (e.g., 1,100 to 1,300 Pa·s).

In some embodiments, the polyethylene copolymers have a shear thinning ratio (η*@0.01/100) less than 15, or in the range of 3 to 15, or 4 to 12, or 5 to 10, or 5.5 to 8.

Rheological data such as “Complex shear viscosity (η*),” reported in Pascal is seconds, can be measured at 0.01 rad/sec and 100 rad/sec. Complex shear viscosity and other rheological measurements can be obtained from small angle oscillatory shear (SAOS) experiments. For instance, complex shear viscosity can be measured with a rotational rheometer such as an Advanced Rheometrics Expansion System (ARES-G2 model) or Discovery Hybrid Rheometer (DHR-3 Model) using parallel plates (diameter=25 mm) in a dynamic mode under nitrogen atmosphere. The rheometer can be thermally stable at 190° C. for at least 20 minutes before inserting compression-molded specimen onto the parallel plates. To determine the specimen's viscoelastic behavior, a frequency sweep in the range from 0.01 to 628 rad/s can be carried out at a temperature of 190° C. under constant strain that does not affect the measured viscoelastic properties. The sweep frequencies are equally spaced on a logarithmic scale, so that 5 frequencies are probed per decade. Depending on the molecular weight and temperature, strains of 3% can be used and linearity of the response is verified. A nitrogen stream is circulated through the oven to minimize chain extension or cross-linking during the experiments. The specimens can be compression molded at 190° C., without stabilizers. A sinusoidal shear strain can be applied. The shear thinning slope (STS) can be measured using plots of the logarithm (base ten) of the dynamic viscosity versus logarithm (base ten) of the frequency. The slope is the difference in the log(dynamic viscosity) at a frequency of 100 s⁻¹ and the log(dynamic viscosity) at a frequency of 0.01 s⁻¹ divided by 4. The complex shear viscosity (η*) versus frequency (ω) curves can be fitted using the Carreau-Yasuda model: η*−η_{∞}=(η₀−η_{∞})*(1+(λω)^{a})^{(n-1)/a}.

The five parameters in this model are: η₀, the zero-shear viscosity; λ, the relaxation time; and n, the power-law index; η_{∞}, the infinite rate viscosity; and a, the transition index. The zero-shear viscosity is the value at a plateau in the Newtonian region of the flow curve at a low frequency, where the dynamic viscosity is independent of frequency. The relaxation time corresponds to the inverse of the frequency at which shear-thinning starts. The power-law exponent describes the extent of shear-thinning, in that the magnitude of the slope of the flow curve at high frequencies approaches n−1 on a log(η*)−log(ω) plot. For Newtonian fluids, n=1 and the dynamic complex viscosity is independent of frequency.

In addition to dynamic and complex viscosity (each in Pascal seconds), at each frequency sweep in the SAOS experiment, various other parameters are collected, including storage modulus (Pa), Loss modulus (Pa), Complex Modulus (Pa), tan(delta), and phase angle. Charting the phase angle versus the complex shear modulus from the rheological experiment yields van Gurp Palmen plots useful to extract some information on the molecular characteristics e.g., linear vs. long chain branched chains, type of long chain branching, polydispersity (Dealy, M. J., Larson, R. G. “Structure and Rheology of Molten Polyners”, Carl Hanser Verlag, Munich 182-183 (2006). It has been also suggested that VGP-plots can be used to reveal the presence of long chain branching in polyethylene. See Trinkle, S., Walter, P., Friedricl, C. “Van (Gurp-Palmen plot II—Classification of long chain branched polymers by their topology”, in 41 Rheol. Acta 103-113 (2002).

“Shear Thinning Ratio”, which is reported as a unitless number, is characterized by the decrease of the complex viscosity with increasing shear rate. Herein, shear thinning can be determined as a ratio of complex viscosity at a frequency of 0.01 rad/s to the complex viscosity at a frequency of 100 rad/s.

A balance of some of the advantageous properties of polyethylene copolymers of the present disclosure may furthermore be represented by an overall property known as a “Q parameter”. Q parameter is represented by Equation 1:

$$Q parameter = 0.001 * {( MIR * {η}_{0.01} / {η}_{100} )}^{2} * ( {T}_{75} - {T}_{25} ) / MWD \qquad ( Eq . 1 )$$

where MIR is melt index ratio, η_{0.001} is complex shear viscosity (η*)@0.01 rad/sec and 190° C., η₁₀₀ is complex shear viscosity (η*)@100 rad/sec and 190° C., T₇₅−T₂₅ is T₇₅−T₂₅ value, where T₂₅ is the temperature at which 25% of the eluted polymer is obtained and T₇₅ is the temperature at which 75% of the eluted polymer is obtained (determined by TREF, as referenced above), and MWD is molecular weight distribution. In some embodiments, polyethylene copolymers of the present disclosure have a Q parameter of 50 to 90, such as 60 to 80, such as 60 to 70, alternatively 70 to 80. Overall, the Q-parameter shows the strong dependence on LCB (˜the 4^{th} power via MIR and viscosity ratio) and the breadth of the co-monomer distribution (through the T₇₅−T₂₅ term); and thus, the Q-parameter captures the uniqueness of the combination of these properties exhibited in the polyethylene copolymers of the present disclosure.

Also or instead, the polyethylene copolymers have a disubstituted vinylene content of about 0.01 to about 0.1 disubstituted vinylenes/1000 carbon atoms, such as about 0.02 to about 0.06 disubstituted vinylenes/1000 carbon atoms. In some embodiments, polyethylene copolymers have a trisubstituted vinylene content of about 0.08 to about 0.2 trisubstituted vinylenes/1000 carbon atoms, such as about 0.1 to about 0.15 trisubstituted vinylenes/1000 carbon atoms. In some embodiments, polyethylene copolymers have a vinyl content of about 0.01 to about 0.05 vinyls/1000 carbon atoms, such as about 0.02 to about 0.04 vinyls/1000 carbon atoms. In some embodiments, polyethylene copolymers have a vinylidene content of is about 0.01 to about 0.05 vinylidenes/1000 carbon atoms, such as about 0.02 to about 0.04 vinylidenes/1000 carbon atoms.

¹H NMR data can be collected at 120° C. in a 10 mm probe using a spectrometer with a ¹H frequency of 600 MHz or higher. Data can be recorded using a maximum pulse width of 45°, 5 seconds between pulses and signal averaging 512 transients. Spectral signals are integrated. Polyethylene copolymer samples can be dissolved in deuterated 1,1,2,2,-tetrachloroethane-d2 at concentrations of 30 mg/ml prior to being inserted into the spectrometer magnet. Prior to data analysis, spectra can be referenced by setting the residual hydrogen-containing solvent resonance to 5.98 ppm. Disubstituted vinylenes can be measured as the number of vinylenes per 1,000 carbon atoms using the resonances between 5.55-5.31 ppm. Trisubstituted vinylenes (“trisubs”) end-groups can be measured as the number of trisubstituted groups per 1,000 carbon atoms using the resonances between 5.3-5.11 ppm, by difference from vinyls. Vinyl end-groups can be measured as the number of vinyls per 1,000 carbon atoms using the resonances between 5.10-4.95 and between 5.3-4.85 ppm. Vinylidene end-groups can be measured as the number of vinylidenes per 1,000 carbon atoms using the resonances between 4.84-4.70 ppm.

As used herein, a “triad” is a three monomer repeat unit: e.g. AAA, AAB, BAA, BAB, ABA, BBA, ABB, BBB summed and normalized to 1. A=CH₂; B=C₆. Triad analysis by ¹³C-NMR gives insight into the sequence distribution and the blockiness of the material. In some embodiments, a polyethylene copolymer has an [EEE]triad content of 88 mol % to 94 mol %, such as 89 mol % to 92 mol %, such as 89 mol % to 91 mol %, as determined by ¹³C nuclear magnetic resonance (¹³C NMR). (“E” is ethylene). In some embodiments, a polyethylene copolymer has an [HEE]triad content of 3 mol % to 8 mol %, such as 4 mol % to 7 mol %, such as 5.3 mol % to 7 mol %, as determined by ¹³C NMR). (“H” is hexene and “E” is ethylene). In some embodiments, a polyethylene copolymer has an [HEH]triad content of 0.05 mol % to 0.5 mol %, such as 0.1 mol % to 0.3 mol %, as determined by ¹³C NMR. In some embodiments, a polyethylene copolymer has an [EHE]triad content of 1 mol % to 5 mol %, such as 2 mol % to 4 mol %, as determined by ¹³C NMR.

For ¹³C NMR samples can be dissolved in deuterated 1,1,2,2-tetrachloroethane-d2 (tce-d2) at a concentration of 67 mg/mL at 140° C. Spectra can be recorded at 120° C. using a Bruker NMR spectrometer of at least 600 MHz with a 10 mm cryoprobe. A 900 pulse, 10 second delay, 512 transients, and gated decoupling can be used for measuring the ¹³C NMR. Polymer resonance peaks are referenced to Polyethylene main peak at 29.98 ppm. Chemical shift assignments for the ethylene based copolymers are described by Randall in “A Review Of High is Resolution Liquid Carbon Nuclear Magnetic Resonance Characterization of Ethylene-Based Polymers”, Polymer Reviews, 29:2, 201-5 317 (1989). The copolymer content, mole and weight %, triad sequencing, and diad calculations are also calculated and described in the method established by Randall in this paper.

### Waste-Processed Olefins

The processing of waste, such as plastic waste, may result in the production or recovery of olefins, or the attribution of waste feedstock to olefins, including any of the alpha-olefins disclosed herein, used in making the polymer compositions (e.g., polyethylene copolymers) disclosed herein. The waste may include plastic waste obtained from any source including, but not limited to, municipal, industrial, commercial or consumer sources. The plastic waste further may be obtained from a common source or from mixed sources, including mixed plastic waste obtained from municipal or regional sources and/or from waste streams of PET, HDPE, LDPE, LLDPE, polypropylene, and/or polystyrene. Furthermore, the waste may include thermoplastic elastomers and thermoset rubbers, such as from tires and other articles made from natural rubber, polybutadiene, styrene-butadiene, butyl rubber and EPDM.

The waste that is processed may also include any of various used polymeric and non-polymeric articles without limitation. Some examples of the many types of polymeric articles may include: films (including cast, blown, and otherwise), sheets, fibers, woven and nonwoven fabrics, fumiture (e.g., garden furniture), sporting equipment, bottles, food and/or liquid storage containers, transparent and semi-transparent articles, toys, tubing and pipes, sheets, packaging, bags, sacks, coatings, caps, closures, crates, pallets, cups, non-food containers, pails, insulation, and/or medical devices. Further examples include automotive, aviation, boat and/or watercraft components (e.g., bumpers, grills, trim parts, dashboards, instrument panels and the like), wire and cable jacketing, agricultural films, geomembranes, playground equipment, and other such articles, whether blow molded, roto-molded, injection-molded, or the like. Any of the foregoing may include mixtures of polymeric and non-polymeric items (e.g., packaging or other articles may include inks, paperboards, papers, metal deposition layers, and the like). The ordinarily skilled artisan will appreciate that such polymeric articles may be made from any of various polymer and/or non-polymer materials, and that the polymer materials may vary widely (e.g., ethylene-based, propylene-based, butyl-based polymers, and/or polymers based on any C₂ to C₄₀ or even higher olefins, and further including polymers based on any one or more types of monomers, e.g., C₂ to C₄₀ α-olefin, di-olefin, cyclic olefin, etc. monomers). Common examples include ethylene, propylene, butylene, pentene, hexene, heptene, and octene; as well as multi-olefinic (including cyclic olefin) monomers such as ethylidene norbomene (ENB) and vinylidene norbomene (VNB) (including, e.g., when such cyclic olefins are used as comonomers, e.g., with ethylene monomers).

Processing of waste, such as through the pyrolysis of plastic waste, may directly produce or recover olefins used to make such polymer compositions or via the attribution of the use of the waste as a feed to a system, such as determined by crediting, allocating, and/or offsetting or substituting for other hydrocarbons in a mass or energy balance for a system, such as in accordance with a third party certification relating to circularity. Polymers that are certified for their circularity by third party certification may be referred to as certified circular. One example of such a certification is the mass balance chain of custody method set forth by the Intemational Sustainability and Carbon Certification.

Various processes may be employed to produce, recover, or attribute to olefins used for the polymers disclosed herein. For example, olefins may be obtained from or in connection with the co-processing of waste, such as plastic waste, with other hydrocarbon feeds in a cracking, coking, hydroprocessing, and/or pyrolysis processes. For example, the olefins may be obtained directly or indirectly from fluid catalytic cracking units, delayed coking units, fluidized coking units (including FLEXICOKING™ units), hydroprocessing units (including hydrocracking and hydrotreating units), and/or steam cracking units (including gas or liquid steam cracking units) receiving such waste as a feed or co-feed. Alternatively, such units may receive a pyrolysis product of the processing of such waste (such as a separated or combined recycle pyrolysis gas and/or recycle pyrolysis oil) as a feed or cofeed. The olefins may be directly produced by such process or may be obtained by further processing, such as separation, treating, and/or cracking of an effluent of such processes. As an example, the olefins may be obtained by the processing of recycle pyrolysis oil and/or recycle pyrolysis gas produced from the pyrolysis of plastic waste. As used herein “recycle pyrolysis oil” refers to compositions of matter that are liquid when measured at 25° C. and 1 atm, and at least a portion of which are obtained from the pyrolysis of recycled waste (e.g., recycled plastic waste). As used herein “recycle pyrolysis gas” refers to compositions of matter that are a gas at 25° C. and 1 atm, and at least a portion of which are obtained from the pyrolysis of recycled waste. In addition, co-processing of waste, such as plastic waste, as a feed or co-feed into fluid catalytic cracking units, delayed coking units, fluidized coking units (including FLEXICOKING™ units), hydroprocessing units (including hydrocracking and hydrotreating units), and/or steam cracking units (including gas or liquid steam cracking units) may result in the attribution of the waste to olefins, polymers, or polymer compositions described herein, such as determined by crediting, allocating, and/or offsetting or substituting for other hydrocarbons in a mass or energy balance for a system, such as in accordance with a third party certification relating to circularity.

Accordingly, processes per various embodiments herein may further include obtaining olefins that have been produced or recovered from the processing of plastic waste or olefins to which the processing of plastic waste has been attributed, e.g., for employment in polymerization processes as elsewhere described herein; and polymer compositions (e.g., polyethylene copolymer) of various embodiments described herein may comprise olefins that have been produced or recovered from the processing of plastic waste or olefins to which the processing of plastic waste has been attributed. As an example, at least a portion of the olefin content (e.g., employed in processes and/or included in compositions as described herein) may be from olefins that are produced or recovered directly from the processing of plastic waste. Similarly, the processing of plastic waste may be attributed to at least a portion the olefins (e.g., employed in processes and/or included in compositions as described herein).

### Blends and Additives

In some embodiments, the polyethylene copolymers can be formulated (e.g., blended) with one or more other polymer components. In some embodiments, those other polymer components are alpha-olefin polymers such as polypropylene or polyethylene homopolymer and copolymer compositions. In some embodiments, those other polyethylene polymers are selected from the group consisting of linear low density polyethylene, high density polyethylene, medium density polyethylene, low density polyethylene, and other differentiated polyethylenes.

In some embodiments, the formulated blends can contain additives, which are determined based upon the end use of the formulated blend. In some embodiments, the additives are selected from the group consisting of fillers, antioxidants, phosphites, anti-cling additives, tackifiers, ultraviolet stabilizers, heat stabilizers, antiblocking agents, release agents, antistatic agents, pigments, colorants, dyes, waxes, silica, processing aids, neutralizers, lubricants, surfactants, and nucleating agents. In some embodiments, additives are present in an amount from 0.1 ppm to 5.0 wt %.

Polyethylene copolymers of the present disclosure can be optionally blended with one or more processing aids to form a polyethylene blend. Because of the improved properties of polyethylene copolymers of the present disclosure, advantageously, such processing aids can be omitted even in blown films (e.g., films, and particularly blown films, of some embodiments may be free of or substantially free of polymer processing aids, and especially polymer is processing aids comprising fluorine; where “substantially free” means free of any intentionally added components, but allowing for up to 100 ppm of such component(s) as impurities).

### Polymerization Processes

The polymerization process can include a gas phase polymerization reaction, and in particular a fluidized bed gas phase polymerization reaction. The gas-phase polymerization may be carried out in any suitable reactor system, e.g., a stirred- or paddle-type reactor system. See U.S. Pat. Nos. 7,915,357; 8,129,484; 7,202,313; 6,833,417; 6,841,630; 6,989,344; 7,504,463; 7,563,851; and 8,101,691 for discussion of suitable gas phase fluidized bed polymerization systems, which are well known in the art.

It has been discovered that polyethylene copolymers of the present disclosure can be formed with a non-sticking polymerization at commercial scale. Appropriate polymerization parameters to prevent sticking in a reactor for formation of polyethylene copolymers of the present disclosure can be determined using a “T_{stick}” method, as described in more detail below and in U.S. Pat. No. 9,718,896 which is incorporated by reference herein.

In such polymerization processes, a gas-phase, fluidized-bed process is conducted by passing a stream containing ethylene and an olefin comonomer continuously through a fluidized-bed reactor under reaction conditions and in the presence of a catalyst composition at a velocity sufficient to maintain a bed of solid particles in a suspended state. A stream (which may be called a “cycle gas” stream) containing unreacted ethylene and olefin comonomer is continuously withdrawn from the reactor, compressed, cooled, optionally partially or fully condensed, and recycled back to the reactor. Prepared polyethylene copolymer is withdrawn from the reactor and replacement ethylene and olefin comonomer are added to the recycle stream. In some embodiments, gas inert to the catalyst composition and reactants is present in the gas stream.

The cycle gas can include induced condensing agents (ICA). An ICA is one or more non-reactive alkanes that are condensable in the polymerization process for removing the heat of reaction. In some embodiments, the non-reactive alkanes are selected from C₁-C₆ alkanes, e.g., one or more of propane, butane, isobutane, pentane, isopentane, hexane, as well as isomers thereof and derivatives thereof In some instances, mixtures of two or more such ICAs may be particularly desirable (e.g., propane and pentane, propane and butane, butane and pentane, etc.).

The reactor pressure may vary from 100 psig (680 kPag)-500 psig (3448 kPag), or in the range of from 200 psig (1379 kPag)-400 psig (2759 kPag), or in the range of from 250 psig (1724 kPag)-350 psig (2414 kPag). In some embodiments, the reactor is operated at a temperature in the range of 60° C. to 120° C., 60° C. to 115° C., 70° C. to 110° C., 70° C. to 95° C., or 80° C. to 90° C. A ratio of hydrogen gas to ethylene can be 10 to 30 ppm/mol %, such as 15 to ppm/mol %, such as 16 to 20 ppm/mol %.

The mole percent of ethylene may be from 25.0-90.0 mole percent, or 50.0-90.0 mole percent, or 70.0-85.0 mole percent, and the ethylene partial pressure is in the range of from 75 psia (517 kPa)-300 psia (2069 kPa), or 100-275 psia (689-1894 kPa), or 150-265 psia (1034-1826 kPa), or 180-200 psia. Ethylene concentration in the reactor can also range from 35-95 mol %, such as within the range from a low of 35, 40, 45, 50, or 55 mol % to a high of 70, 75, 80, 85, 90, or 95 mol % and further where ethylene mol % is measured on the basis of total moles of gas in the reactor (including, if present, ethylene and/or comonomer gases as well as inert gases such as one or more of nitrogen, isopentane or other ICA(s), etc.); as with vol-ppm hydrogen, this measurement may for convenience be taken in the cycle gas outlet rather than in the reactor itself Comonomer concentration can range from 0.2-1.0 mol %, such as within the range from a low of 0.2, 0.3, 0.4 or 0.5 mol % to a high of 0.65, 0.70, 0.75, 0.80, 0.85, 0.90, 0.95, or 1.0 mol %.

### Catalysts

The catalysts employed in the polymerization can be metallocene catalysts. In particular, metallocene catalysts may be selected from the catalysts described in Patent Cooperation Treaty Publication Nos. WO1993008221, WO1996008520, WO1998044011, and WO2007130277, incorporated herein by reference for all purposes. For instance, the catalysts may be silica-supported metallocene catalyst prepared from compositions comprising dimethylsilylbis(tetrahydroindenyl) zirconium dichloride metallocene and methylalumoxane cocatalyst. In some embodiments, a catalyst is dimethylsilylbis(tetrahydroindenyl) zirconium dichloride.

### Non-Sticking Polymerizations

Described herein are systems and methods for determining a non-sticking operating regime (a “safe” regime) for a polymerization reactor, and operating the polymerization reactor within the non-sticking regime, in particular to obtain polyethylene copolymers in accordance with the above description. As used herein, a non-sticking operating regime indicates a regime in which resin sticking to component(s) of a reactor is not problematic. The methods may include developing a model of the non-sticking operating regime which may be integrated into a control system or used on a separate system to control reaction parameters; controlling the polymerizing using said model; and obtaining a polyethylene copolymer in accordance with the above description.

The parameters used in developing a model of the non-sticking operating regime may be based on values measured during experimental determinations of resin stickiness. For a single target resin, resin sticking can be measured as a function of temperature and the equivalent partial pressure of an induced condensing agent (referred to as an “ICA”). For example, measuring may be performed by placing the resin in a stirred autoclave reactor with a measured amount of an ICA, such as isopentane (IC5), and slowly increasing the temperature until the resin sticks, causing the stirrer to stall. A model can then be built that predicts sticking temperature as a function of the reactor temperature and an equivalent partial pressure of the ICA. The equivalent partial pressure is used to account for other condensable materials that may be present in the reactor, such as hexene and various isomers of hexene. The model is generally specific to the type of resin used.

The model and dew point of the ICA used in the polymerization reaction are used to determine a non-sticking operating regime. During the polymerization reaction, the reactor is controlled to hold the temperature and ICA concentration within the non-sticking operating regime. The non-sticking operating regime can provide guidance to help maximize production rates without agglomeration by controlling the reaction parameters to allow for increasing both temperature and ICA content, thus allowing the removal of more heat of reaction.

As used herein, the term “diluent” (or “condensable diluent” or “condensable diluent gas”) denotes condensable gas (or a mixture of condensable gases) present in a polymerization reactor with polymer resin being produced. The diluent is condensable at the temperatures encountered in the process heat exchanger. Examples of diluents include induced condensing agents (ICAs), comonomers, isomers of comonomers, and combinations thereof. Such materials can include isobutane, isopentane, hexene, and other materials in the reactor.

Stickiness tests can be conducted on a sample resin in a testing device, as described in U.S. Pat. No. 9,718,896, to gain a better understanding of the operability window in resin production with various metallocene catalysts. Through tests on a number of catalysts it can be determined that unique parameters could be developed for each of a number of resins made using different catalysts. The stickiness risk associated with the resin made with these catalysts could be reduced by using a combination of temperature, MI/density/MFR targets, ethylene partial pressure, induced condensing agent (e.g., IC5 or isohexane) concentration, and continuity additives.

A testing apparatus may be used to measure stickiness temperature. The apparatus can use an autoclave reactor that has a mixing motor. The mixing motor rotates a mixer blade that is inserted into a bed of resin in the autoclave. The temperature in the autoclave is slowly is raised until the torque required to turn the mixer blade overcomes the torque available from the mixing motor, and the mixer blade stops rotating, indicating the temperature at which the resin sticks or agglomerates. An illustrative mixing motor that may be used is an air driven motor Model #2AM-NCC-16, manufactured by Gast Manufacturing, Inc. The mixing motor turns a magnetic coupler, which in turn spins the mixer blade. An illustrative magnetic coupler that may be used is a MagneDrive®2, manufactured by Autoclave Engineers.

The testing device can run the stickiness experiments at dry conditions, and also in the presence of induced condensing agents, such as isopentane (IC5) and isohexane (IC6). Although details are presented for a specific testing apparatus, it will be understood that any device capable of consistently measuring the torque of a rotating mixer blade can be used to develop the model for a particular resin.

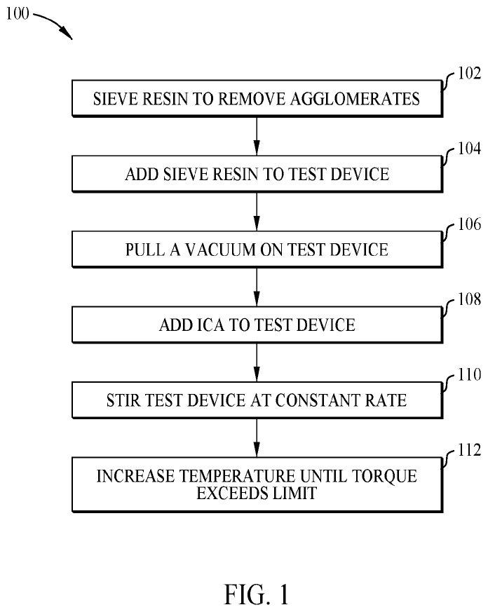

FIG. 1 is a process flow diagram showing a method 100 for measuring stickiness temperature. The method 100 may be used, for example, with the testing device. The method 100 begins at block 102 with the sieving of a resin sample. The sieving removes agglomerates that can interfere with the stickiness measurements. For example, the resin sample can be sieved through a number 12 mesh (having about 1.68 mm openings). At block 104, a measured amount of the resin is added to the testing device. For example, about 300 g of sieved polymer resin can be added to the testing device. At block 106, the testing device is placed under a vacuum prior to adding an ICA, such as IC5, to ensure proper measurement of the partial pressure of the ICA. At block 108, an amount of ICA is added to the testing device to reach a predicted partial pressure. For example, using the testing device, five levels are tested for each resin tested, corresponding to 0, about 25 cc, about 50 cc, about 100 cc, or about 200 cc of added IC5. At block 110, the testing device is then stirred at a constant rate. For example, using the air-operated stirring motor of the testing device, a constant nitrogen pressure of about 30 psi (about 207 kPa) is applied to hold a constant torque.

At block 112, the reactor temperature is increased slowly until a torque limit is exceeded. For example, using the testing device, when the torque limit is exceeded the mixing motor stops, indicating the stickiness temperature. The testing is not limited to the stopping of an air-operated mixing motor. For example, a torque measurement device may be used to measure the torque applied to the testing device to determine when the torque exceeds a preset target.

For most of the test, the mixer speed is substantially constant. However, as the resin starts to agglomerate, the mixer speed starts to slow before stopping. The point at which the mixer speed drops to zero is the stickiness temperature. The test is repeated at a number of different addition levels of ICA, as noted above.

The stickiness temperature is correlated as a linear function of ICA concentration. The results from the testing allow the development of a model to predict the resin sticking temperature (Tstick) that encompasses the metallocene catalyst systems tested. The coefficients of the linear functions can be generated as a function of resin density, MI and MFR. Although the test resins can be made using metallocene catalysts, as the model is empirically generated, the parameters may be adjusted for other catalyst systems, for example, by repeating the model development runs for those resins.

The model predictions can be validated against bed settling experiments done in a pilot plant scale, gas-phase fluidized bed reactor. In these experiments, a non-reacting run can be performed to determine the temperatures at which the resin agglomerates. The test can be started by drying the reactor with a high purity nitrogen purge at elevated temperatures, e.g., greater than about 75° C. The test resin sample can be passed through a 10-mesh screen (having about 0.25 mm openings) to remove agglomerates and then charged to the reactor. Using the nitrogen flow, the resin can be dried to about 10 parts-per-million by volume (ppmv) of water. The test resin can be heated to at least 85° C. and the reactor conditions can be adjusted to the desired ethylene partial pressure, comonomer concentration, and ICA concentration. A sample can then be collected for measurement of melt flow and particle size.

The resin temperature can then be increased by about 2° C. or 3° C. at a rate of about 1° C. every 30 minutes. Once the target temperature is reached, the temperature is allowed to stabilize for 30 minutes. The fluidized bulk density, bed weight, and skin temperature can be noted. The circulation compressor can then be turned off, and the bed allowed to settle on the distributor plate. After about 15 minutes, the circulation compressor can be turned back on to fluidize the resin. If the bed does not fluidize, the test is ended. If the bed does fluidize, the reactor is given about five minutes to stabilize before initiating the next increase in temperature. The procedure is repeated until the bed agglomerated to the point that fluidization is lost.

The stickiness temperature model can be combined with dew point calculations to define an operability window. e.g., a non-sticking operating regime in a map of reactor operations, for the manufacturing of resins made with tested metallocene catalysts. Other models may be created that are specific to resins made by other metallocene catalysts, Ziegler catalysts, or chromium catalysts, among others. As the model is based on the empirical measurements of resin properties and reactor conditions, resins generated from mixtures and combinations of catalysts may also be made.

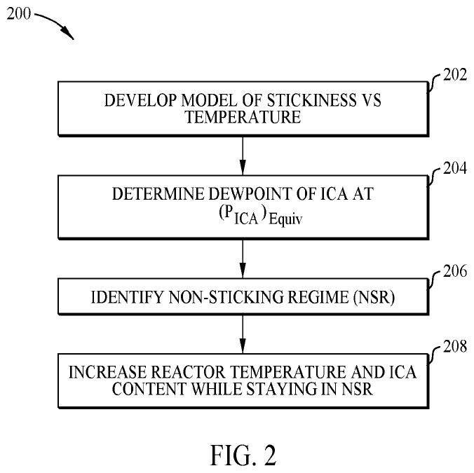

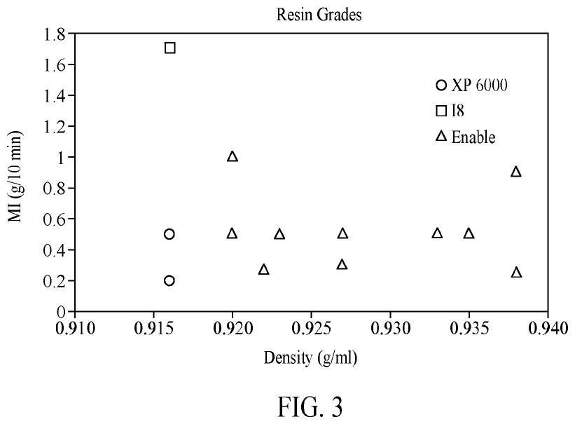

FIG. 2 is a process flow diagram of a method 200 for operating a reactor in a non-sticking regime. The method 200 starts at block 202 with the development of a model for the stickiness temperature. The model may be developed, for example, using measurements made with the method 100 discussed with respect to FIG. 1, and fitting the measured data to develop parameters for Eqns. 1-4. At block 204, a dew point for the ICA can be determined at each of the equivalent partial pressures for the ICA. The dew point indicates the conditions of temperature and equivalent partial pressures of ICA below which liquid ICA starts to condense in the reactor. The formation of liquid ICA can increase the likelihood of agglomeration and case operational issues by condensing in instrumentation taps.

At block 206, the stickiness temperature and the dew point can be used to identify a non-sticking regime. Once a non-sticking regime is established, at block 208 the ICA concentration and temperature can be adjusted to remain in the safe operating regime. For example, a startup of a new resin production run may be conducted at a slow initial production rate. The ICA concentration, temperature, or both may then be slowly increased to increase the production rate, while keeping the reactor within the safe operating regime. If a reactor upset causes operations to leave the non-sticking regime, or indicates that sticking may be imminent, the control system can recommend changes to force the operations back into the non-sticking regime, for example, by lowering or raising the temperature, by decreasing the amount of ICA returned from the recycle system, or by injecting a kill solution to slow or stop the reaction, among others. The control system may identify problematic operations before the reactor is shut down by agglomeration.

The predicted stickiness temperature (Tstick) from the model can be plotted. To provide a limit, the Tstick is adjusted to a lower value to provide a safety margin, using Eqn. 2.

$$Treactor , max = Tstick - {UT}_{Dmax} \qquad Eqn . 2$$

In Eqn. 2, Treactor,max represents the maximum operating temperature that can be used without a substantial risk of agglomeration. UT_{Dmax} represents an upper temperature delta that provides a buffer between the stickiness temperature measured in the experiments and the temperature at which the stickiness may actually begin. Typically, a 10° C. margin is allowed below the stickiness temperature for the reactor to operate safely. Thus, the value of the Treactor,max provides the upper temperature limit for the reactor.

The dew point (Tdew) can be plotted. Similar to the maximum operating temperature, the dew point can be adjusted to provide a wider margin of safety using Eqn. 3.

$$Treactor , min = {T}_{d e w} + L {T}_{Dmax} \qquad Eqn . 3$$

In Eqn. 3, LT_{Dmax} is a lower temperature delta that accounts for capillary condensation, which occurs about 10° C. above the actual dew point of the ICA in the reactor. The value of the Treactor,min provides the lower temperature limit of the reactor.

The upper temperature limit and the lower temperature limit define a non-sticking regime for the reactor within the two dimensional space plotted. Another area defined by these limits is a sticking regime in which the resin begins to melt and therefore becomes sticky. Other areas include a stick+liquid regime, in which both resin melting and IC5 (or other ICA) condensation make the resin sticking more likely. Below the upper temperature limit and the lower temperature limit is a liquid regime, in which the IC5 (or other ICA) starts to condense and make the resin sticky.

A current reactor condition can be mapped by the temperature and equivalent partial pressure of the ICA. To operate the reactor without agglomeration, an operator maintains the current reactor conditions within the non-sticking regime. The operator can change reactor parameters to move the reactor conditions towards the neck, at which the limits meet, to increase productivity, while still staying within the non-sticking regime. It can be noted that as the conditions are pushed closer to the neck, operations becomes less flexible and the room for error dwindles, making process upsets, such as temperature and concentration excursions, more problematic.

The model substantially predicts the operating window both in a pilot plant reactor and in a commercial plant. The model (e.g., equation determined through testing per above) can be used to produce polyethylene copolymer (e.g., in accordance with those described herein), for instance by setting desirable operating conditions to provide a desired production rate by increasing the ICA concentration while still remaining in the non-sticking regime. Further, the model can be used to identify operations in problematic regimes, and adjust the reactor conditions before operational problems or shut-downs occur.

### Articles of Manufacture

The polyethylene copolymers of the present disclosure can be particularly suitable for making end-use articles of manufacture such as films (e.g., as may be formed by lamination, extrusion, coextrusion, casting, and/or blowing); as well as other articles of manufacture as may be formed, e.g., by rotomolding or injection molding. Polyethylene copolymers can be formed into articles of manufacture by cast film extrusion, blown film extrusion, rotational molding or injection molding processes. In some embodiments, the polyethylene copolymer can be used in a blend.

It has been discovered that polyethylene copolymers of the present disclosure can provide excellent shear thinning characteristics with little or no melt fracture of the extrudate at high die shear rates. For example, polyethylene copolymers of the present disclosure having an MI of 1.5 to 2.5 and a density of about 0.92 g/cm³ (such as 0.918 to 0.921 g/cm³ or 0.916 to 0.921 g/cm³) can be used as skin layers in multilayer blown films to provide melt fracture free films, which allows polymer processing aids (such as per- or poly-fluoro alkanes) to be merely optional (or, preferably, eliminated entirely from such blown film formulations, such that the films are free or substantially free of polymer processing aids such as fluorine-containing polymer processing aids). Further, polyethylene copolymers of the present disclosure (e.g., MI of 1.5 to 2.5 and a density of 0.914 to 0.925 g/cm³, such as 0.916 to 0.921 g/cm³) can provide films formed with reduced motor load and melt pressure (which increases input) due to improved flow behavior, as compared to other LLDPEs.

In some embodiments, a polyethylene copolymer or blend thereof provides a smooth extrudate at an apparent die (wall) shear rate between 80-150 s⁻¹ without significant melt fracture by visual observation (again, even without PPA such as fluorine-containing PPA).

A polyethylene copolymer (or blend thereof) of the present disclosure can be useful in such forming operations as film, sheet, and fiber extrusion and co-extrusion as well as blow molding, injection molding, and rotary molding. Films include blown or cast films formed by co-extrusion or by lamination useful as shrink film, cling film, stretch film, sealing films, oriented films, snack packaging, heavy duty bags, grocery sacks, baked and frozen food packaging, medical packaging, industrial liners, membranes, etc., in food-contact and non-food contact applications. Fibers include melt spinning, solution spinning and melt blown fiber operations for use in woven or non-woven form to make filters, diaper fabrics, medical garments, geotextiles, etc. Extruded articles include medical tubing, wire and cable coatings, pipe, geomembranes, and pond liners. Molded articles include single and multi-layered constructions in the form of bottles, tanks, large hollow articles, rigid food containers and toys, etc.

The polyethylene copolymers (or blends thereof) may be formed into monolayer or multilayer films. These films may be formed by any of the conventional techniques including extrusion, co-extrusion, extrusion coating, lamination, blowing and casting. The film may be obtained by the flat film or tubular process which may be followed by orientation in a uniaxial direction or in two mutually perpendicular directions in the plane of the film. One or more of the layers of the film may be oriented in the transverse and/or longitudinal directions to the same or different extents. This orientation may occur before or after the individual layers are brought together. For example a polyethylene copolymer (or blend thereof) layer can be extrusion coated or laminated onto an oriented polypropylene layer or the polyethylene copolymer (or blend thereof) and polypropylene can be coextruded together into a film then oriented. Likewise, oriented polypropylene could be laminated to oriented polyethylene copolymer (or blend thereof), or oriented polyethylene copolymer (or blend thereof) could be coated onto polypropylene then optionally the combination could be oriented even further.

Films include monolayer or multilayer films. Specific end use films include, for example, blown films, cast films, stretch films, stretch/cast films, stretch cling films, stretch handwrap films, machine stretch wrap, shrink films, shrink wrap films, greenhouse films, laminates, and laminate films. Exemplary films are prepared by any conventional technique known to those skilled in the art, such as for example, techniques utilized to prepare blown, extruded, and/or cast stretch and/or shrink films (including shrink-on-shrink applications).

In one embodiment, multilayer films (multiple-layer films) may be formed by any suitable method. The total thickness of multilayer films may vary based upon the application desired. A total film thickness of 5-100 μm, such as 10-50 μm, is suitable for most applications. Those skilled in the art will appreciate that the thickness of individual layers for multilayer films may be adjusted based on desired end-use performance, polymer(s) employed, equipment capability, and other factors. The materials forming each layer may be coextruded through a coextrusion feedblock and die assembly to yield a film with two or more layers adhered together but differing in composition. Coextrusion can be adapted for use in both cast film or blown film processes. Exemplary multilayer films have at least two, at least three, or at least four layers. In one embodiment the multilayer films are composed of five to ten layers.

### EXAMPLES

### Example 1

Multiple experiments to explore a new combination of melt index and density to understand product performance were conducted in a gas phase fluidized bed polymerization reactor.

A combination of resin properties were chosen as a suitable candidate for commercial-scale manufacturing. Historic modeling calculations were performed utilizing a method based on a manufacturing reactor environment. The results of the initial calculations determined there was a high risk of discontinuity in a commercial reactor environment. In the absence of further information, these modeling calculations alone might have led to discontinuing the project. However, the Tstick model, new to this type of metallocene catalyzed LLDPEs, was also applied to calculate the continuity risk, serving as an additional check on the legacy modeling. It was then determined that the risk was not as high as initially anticipated, which resulted in additional pilot plant testing. Further experiments were executed on pilot-scale reactors to understand full product performance at various temperatures, since temperature can be a contributor to poor continuity and stickiness.

Polyethylene copolymers, according to one or more embodiments provided herein, were produced in gas phase polymerization systems. Properties of the inventive resins are shown in Table 1.

Inventive resins I1, I2, I3, I4, I5 and I13 were produced at pilot plant scale; inventive resin I8 was produced at commercial scale.

The polymerization was conducted in a continuous gas phase fluidized bed reactor. Polymerization conditions are shown below in Table 2. The fluidized bed was made up of catalyst and growing polymer granules. The gaseous feed streams of ethylene and hydrogen together with liquid comonomer were mixed together in a mixing tee arrangement and introduced below the reactor bed into the recycle gas line. An ICA (isopentane) was added with the ethylene and hydrogen and also introduced below the reactor bed into the recycle gas line. The individual flow rates of ethylene, hydrogen and comonomer were controlled to maintain fixed composition targets. The ethylene concentration was controlled to maintain a constant ethylene partial pressure. The hydrogen was controlled to maintain a constant hydrogen to ethylene mole ratio. The concentration of each gas was measured by an on-line gas chromatograph to ensure relatively constant composition in the recycle gas stream.

Commercial ENABLE™ 2010 polyethylene grade exhibits flow challenges in the die and extruder used in Cast Film lines, causing high melt pressures, high motor load, and suboptimal flow to edge in the die, which can result in adjacent resin layer encapsulation. As shown in FIG. 1, the inventive resins are outside of the Enable window of MI and density. As shown in FIG. 1, “Enable” refers to various ENABLE™ resin grades available from ExxonMobil Chemical Company of Houston, TX. “XP 6000” is a series of resins available from ExxonMobil Chemical Company of Houston, TX. 18 is an example of an inventive resin.

A higher melt index of 18 resin as compared to ENABLE™ 2010 was found to improve the flow behavior and ameliorate process limitations. A lower density (e.g., 0.916 g/cm³ and 0.918 g/cm³) was found to improve film properties such as puncture; achieving such an improved (i.e., higher) puncture improves wrapping consistency and can improve holding force on a pallet during transportation.

In addition, the presence of a small amount of long chain branches (LCB) in commercial ENABLE™ grades can improve processability in cast film application. The presence of LCB generally provides processing advantages such as reduced neck-in and increased draw stability. It was discovered that inventive resins of the present disclosure can maintain such advantageous LCB, even while achieving the improved density and MI, surprisingly achieving acceptable rates of production suitable for commercial processes. In addition, the lower zero shear viscosity of inventive resins compared to the comparative commercial grades can lead to lower head pressures and temperatures during extrusion, which can lead to increased output.

TABLE 1.1
Analytical data of inventive resins I1-I8

| | | | | | | | | |
|---|---|---|---|---|---|---|---|---|
| Sample ID | I1 | I2 | I3 | I4 | I5 | I6 | I7 | I8 |

MI and Density

| | | | | | | | | |
|---|---|---|---|---|---|---|---|---|
| MI (g/10 min) | 1.60 | 1.81 | 1.97 | 2.20 | 2.29 | 1.74 | 1.77 | 1.78 |
| HLMI (g/10 min) | 49.5 | 55.4 | 60.6 | 63.8 | 67.1 | 49.5 | 51.2 | 50.3 |
| MIR | 31 | 31 | 31 | 29 | 29 | 28 | 29 | 28 |
| Base Resin | 0.921 | 0.921 | 0.920 | 0.919 | 0.916 | 0.916 | 0.917 | 0.916 |
| density (g/ml) | | | | | | | | |

GPC-4D

| | | | | | | | | |
|---|---|---|---|---|---|---|---|---|
| Mₙ (g/mol) | 25000 | 24000 | 24000 | 22000 | 23000 | 25000 | 25000 | 24000 |
| M_{w} (g/mol) | 83000 | 81000 | 79000 | 76000 | 77000 | 87000 | 86000 | 85000 |
| MWD | 3.4 | 3.4 | 3.3 | 3.5 | 3.4 | 3.4 | 3.5 | 3.6 |
| M_{z} | 168000 | 164000 | 161000 | 154000 | 157000 | | | 175000 |
| M_{z+1} | 268000 | 259000 | 257000 | 242000 | 254000 | | | 279000 |
| LCB, g′ | 0.94 | 0.97 | 0.96 | 0.96 | 0.96 | 0.96 | 0.97 | 0.96 |

SAOS

| | | | | | | | | |
|---|---|---|---|---|---|---|---|---|
| η_{0.01} (Pas) | 9400 | 800 | 6800 | 6700 | 6500 | 7900 | 7700 | 7700 |
| η₁₀₀ (Pas) | 1300 | 1200 | 1100 | 1100 | 1100 | 1200 | 1200 | 1200 |
| η_{0.01}/η₁₀₀ | 7.2 | 6.7 | 6.2 | 6.1 | 5.9 | 6.6 | 6.4 | 6.4 |

¹³C-NMR (Triad)

| | | | | | | | | |
|---|---|---|---|---|---|---|---|---|
| EEE (mol %) | 0.918 | 0.916 | 0.911 | 0.906 | 0.894 | 0.892 | 0.899 | 0.894 |
| HEE | 0.053 | 0.054 | 0.057 | 0.060 | 0.069 | 0.068 | 0.065 | 0.067 |
| HEH | 0.001 | 0.001 | 0.001 | 0.002 | 0.002 | 0.002 | 0.002 | 0.002 |
| EHH | 0.001 | 0.000 | 0.001 | 0.001 | 0.002 | 0.002 | 0.002 | 0.002 |
| HHH | 0.001 | 0.002 | 0.000 | 0.001 | 0.000 | 0.000 | 0.000 | 0.000 |
| C₂ (mol %) | 97.2 | 97.1 | 97 | 96.8 | 96.4 | 96.3 | 96.6 | 96.4 |
| C₆ (mol %) | 3.2 | 3.6 | 2.8 | 3.2 | 3.6 | 3.7 | 3.4 | 3.6 |
| C₂ (wt %) | 92.0 | 91.8 | 91.5 | 90.9 | 90.0 | 89.7 | 90.4 | 89.8 |
| C₆ (wt %) | 8.0 | 8.2 | 8.5 | 9.1 | 10.0 | 10.3 | 9.6 | 10.2 |
| Branches/1000 C | 13.3 | 13.6 | 14.1 | 15.1 | 16.7 | 17.4 | 16.1 | 17.0 |

1H-NMR

| | | | | | | | | |
|---|---|---|---|---|---|---|---|---|
| Hexene, wt % | 8.2 | 7.9 | 8.5 | 8.9 | 10.1 | 9.9 | 9.6 | 10.0 |
| Vinylenes/1000 C | 0.05 | 0.04 | 0.04 | 0.03 | 0.05 | | | |
| Trisubstituted | 0.13 | 0.13 | 0.10 | 0.10 | 0.14 | | | |
| olefins/1000 C | | | | | | | | |
| Vinyls/1000 C | 0.03 | 0.02 | 0.02 | 0.02 | 0.03 | | | |
| Vinylidenes/ | 0.03 | 0.03 | 0.02 | 0.03 | 0.04 | | | |
| 1000 C | | | | | | | | |

TREF

| | | | | | | | | |
|---|---|---|---|---|---|---|---|---|
| CDBI (%) | 85.3 | 85.0 | 84.3 | 85.3 | 83.2 | 85.2 | 85.0 | 85.5 |
| T_{w} (° C.) | 77.6 | 77.1 | 75.6 | 74.9 | 72.0 | 72.5 | 73.5 | 72.4 |
| T_{med} (° C.) | 80.0 | 79.6 | 78.5 | 77.4 | 75.0 | 75.3 | 76.2 | 75.2 |
| Tₚₑₐₖ (° C.) | 81.3 | 80.8 | 79.9 | 78.8 | 77.0 | 76.9 | 77.7 | 76.8 |
| T₇₅ − T₂₅ (° C.) | 5.9 | 6.1 | 6.7 | 6.7 | 8.5 | 7.5 | 8.0 | 7.4 |

Q Parameter

| | | | | | | | | |
|---|---|---|---|---|---|---|---|---|
| (0.001*MIR * | 87 | 74 | 73 | 60 | 75 | 77 | 79 | 67 |
| η_{0.01}/η₁₀₀)² * | | | | | | | | |
| (T₇₅ − T₂₅/MWD) | | | | | | | | |

TABLE 1.2
Analytical data of inventive resins I9-I13

| | | | | | |
|---|---|---|---|---|---|
| Sample ID | I9 | I10 | I11 | I12 | I13 |

MI and Density

| | | | | | |
|---|---|---|---|---|---|
| MI (g/10 min) | 1.81 | 1.78 | 1.76 | 1.75 | 1.75 |
| HLMI (g/10 min) | 51.5 | 49.4 | 50.5 | 49.6 | 48.5 |
| MIR | 29 | 28 | 29 | 28 | 28 |
| Base Resin | 0.916 | 0.916 | 0.916 | 0.916 | 0.915 |
| density (g/ml) | | | | | |

GPC-4D

| | | | | | |
|---|---|---|---|---|---|
| Mₙ (g/mol) | 25000 | 24000 | 24000 | 24000 | 21000 |
| M_{w} (g/mol) | 85000 | 85000 | 85000 | 85000 | 80000 |
| MWD | 3.5 | 3.55 | 3.5 | 3.5 | 3.8 |
| M_{z} | | | | | 179000 |
| M_{z+1} | | | | | 317000 |
| LCB, g′ | 0.96 | 0.98 | 0.97 | 0.96 | 0.96 |

SAOS

| | | | | | |
|---|---|---|---|---|---|
| η_{0.01} (Pas) | 7400 | 7700 | 7900 | 8100 | 8100 |
| η₁₀₀ (Pas) | 1200 | 1200 | 1200 | 1200 | 1300 |
| η_{0.01}/η₁₀₀ | 6.2 | 6.4 | 6.6 | 6.8 | 6.2 |

¹³C-NMR (Triad)

| | | | | | |
|---|---|---|---|---|---|
| EEE (mol %) | 0.897 | 0.896 | 0.893 | 0.894 | 0.893 |
| HEE | 0.066 | 0.067 | 0.068 | 0.057 | 0.068 |
| HEH | 0.002 | 0.002 | 0.002 | 0.002 | 0.002 |
| EHE | 0.033 | 0.034 | 0.034 | 0.034 | 0.035 |
| EHH | 0.002 | 0.002 | 0.002 | 0.002 | 0.002 |
| HHH | 0.000 | 0.000 | 0.000 | 0.000 | 0.000 |
| C₂ (mol %) | 96.5 | 96.4 | 96.3 | 96.4 | 96.3 |
| C₆ (mol %) | 3.5 | 3.6 | 3.7 | 3.6 | 3.7 |
| C₂ (wt %) | 90.1 | 90.0 | 89.8 | 89.8 | 89.7 |
| C₆ (wt %) | 9.9 | 10.0 | 10.2 | 10.2 | 10.3 |
| Branches/1000 C | 16.5 | 16.7 | 17.0 | 17.0 | 17.2 |

1H-NMR

| | | | | | |
|---|---|---|---|---|---|
| Hexene, wt % | 9.8 | 10.1 | 10.0 | 10.0 | 9.8 |
| Vinylenes/1000 C | | | | | 0.05 |
| Trisubstituted | | | | | 0.05 |
| olefins/1000 C | | | | | |
| Vinyls/1000 C | | | | | 0.02 |
| Vinylidenes/1000 C | | | | | 0.00 |

TREF

| | | | | | |
|---|---|---|---|---|---|
| CDBI (%) | 85.6 | 85.6 | 85.56 | 85.6 | 86.6 |
| T_{w} (° C.) | 72.6 | 72.7 | 72.7 | 72.5 | 72.6 |
| T_{med}(° C.) | 75.4 | 75.4 | 75.4 | 75.2 | 74.7 |
| Tₚₑₐₖ (° C.) | 77.0 | 77.0 | 77 | 76.8 | 76.0 |
| T₇₅ − T₂₅ (° C.) | 7.4 | 7.3 | 7.3 | 7.4 | 6.7 |

Q Parameter

| | | | | | |
|---|---|---|---|---|---|
| (0.001*MIR * | 65 | 65 | 74 | 78 | 53 |
| η_{0.01}/η₁₀₀)² * | | | | | |
| (T₇₅ − T₂₅/MWD) | | | | | |

TABLE 1.3
Analytical data of comparative resins C1-C7

| Sample ID | C1 | C2 | C3 | C4 | C5 | C6 | C7 |
|---|---|---|---|---|---|---|---|
| Description | Enable ™ | Enable ™ 3505 | Enable ™ 4009 | Enable ™ 4002 | Exceed ™ | Exceed ™ | TOTAL ™ |
| | 2010ME | | | | 2018MA | XP | M1810EP |
| | | | | | | 8318ML | |

MI and Density

| | | | | | | | |
|---|---|---|---|---|---|---|---|
| MI (g/10 min) | 1.60 | 0.5 | 0.9 | 0.2 | 1.7 | 1.11 | 1.2 |
| HLMI | 33.7 | 24.11 | 36.85 | 13.5 | 26.9 | 32.3 | 25.1 |
| (g/10 min) | | | | | | | |
| MIR | 35 | 48 | 41 | 68 | 16 | 29 | 20.9 |
| Base Resin | 0.925 | 0.935 | 0.940 | 0.940 | 0.920 | na | na |
| density (g/ml) | | | | | | | |

GPC-4D

| | | | | | | | |
|---|---|---|---|---|---|---|---|
| Mₙ (g/mol) | 26000 | 27000 | 21000 | 27000 | 37000 | 28000 | 39000 |
| M_{w} (g/mol) | 90000 | 107000 | 88240 | 109000 | 94000 | 108000 | 104000 |
| MWD | 3.5 | 3.9 | 4.2 | 4.1 | 2.6 | 3.8 | 2.7 |
| M_{z} | 186000 | 250000 | 198000 | 257000 | 168000 | 276000 | 182000 |
| M_{z+1} | 296000 | 408000 | 322000 | 426000 | 260000 | 511000 | 285000 |
| LCB, g′ | 0.98 | 0.98 | 0.99 | 0.97 | 1.0 | 0.98 | 1.01 |

SAOS

| | | | | | | | |
|---|---|---|---|---|---|---|---|
| η_{0.01} (Pas) | 19000 | 76300 | 19300 | 134400 | 4600 | 7300 | 8600 |
| η₁₀₀ (Pas) | 1400 | 1800 | 1600 | 2190 | 2000 | 1500 | 1700 |
| η_{0.01}/η₁₀₀ | 13.6 | 42.4 | 12.0 | 61.4 | 2.3 | 4.9 | 5.1 |

¹³C-NMR (Triad)

| | | | | | | | |
|---|---|---|---|---|---|---|---|
| EEE (mol %) | 0.920 | 0.973 | 0.979 | 0.983 | 0.923 | na | na |
| HEE | 0.052 | 0.017 | 0.014 | 0.012 | 0.050 | | |
| HEH | 0.001 | 0.000 | 0.000 | 0.000 | 0.001 | | |
| EHE | 0.026 | 0.009 | 0.007 | 0.005 | 0.024 | | |
| EHH | 0.001 | 0.000 | 0.000 | 0.000 | 0.001 | | |
| HHH | 0.000 | 0.001 | 0.001 | 0.001 | 0.001 | | |
| C₂ (mol %) | 97.3 | 99.1 | 99.3 | 99.5 | 97.4 | | |
| C₆ (mol %) | 2.7 | 0.9 | 0.7 | 0.5 | 2.6 | | |
| C₂ (wt %) | 92.3 | 97.3 | 97.9 | 98.4 | 92.6 | | |
| C₆ (wt %) | 7.7 | 2.7 | 2.1 | 1.6 | 7.4 | | |
| Branches/1000 C | 12.8 | 4.6 | 3.5 | 2.7 | 12.3 | | |

1H-NMR

| | | | | | | | |
|---|---|---|---|---|---|---|---|
| Hexene, wt % | | 3.1 | | | | | |
| Vinylenes/1000 C | 0.03 | 0.07 | 0.06 | na | | na | |
| Trisubstituted | 0.09 | 0.07 | 0.05 | | | | |
| olefins/1000 C | | | | | | | |
| Vinyls/1000 C | 0.02 | 0.02 | 0.02 | | | | |
| Vinylidenes/1000 C | 0.02 | 0.00 | 0.00 | | | | |
| CryoTREF | | | | | | | |
| CDBI (%) | 83.5 | 86.4 | na | na | 70.2 | 39.6 | 7.2 |
| T_{w} (° C.) | 78.0 | 89.8 | | 91.6 | 79.9 | 78.1 | 81.7 |
| T_{med} (° C.) | 80.7 | 91.2 | | 93.2 | 81.6 | 76.9 | 84.2 |
| Tₚₑₐₖ (° C.) | 82.0 | 91.6 | | 93.4 | 83.3 | 69.0 | 86.5 |
| T₇₅ − T₂₅ | 6.1 | 2.0 | | 1.4 | 10.1 | 25.1 | 6.6 |
| (° C.) | | | | | | | |

Q Parameter

| | | | | | | |
|---|---|---|---|---|---|---|
| (0.001*MI | 382 | 2143 | 5772 | 5 | 133 | 27 |
| R * | | | | | | |
| η_{0.01}/η100)² | | | | | | |
| * | | | | | | |
| (T₇₅ − T₂₅/ | | | | | | |
| MWD) | | | | | | |

TABLE 2
Process conditions for certain inventive examples

| | I1 | I2 | I3 | I4 | I5 | I8 | I13 |
|---|---|---|---|---|---|---|---|
| Residence Time (h) | 4.8 | 5.2 | 5.3 | 6.2 | 4.5 | 1.8 | 5.1 |
| Production Rate | 153 | 141 | 138 | 120 | 160 | 79,200 | 137 |
| (lbs/hr) | | | | | | | |
| Reactor Bed Temp, | 188 | 188 | 188 | 180 | 188 | 180 | 188 |
| Median (° F.) | | | | | | | |
| Ethylene Partial | 195 | 195 | 194 | 190 | 190 | 190 | 189 |
| Pressure (PSIA) | | | | | | | |
| Hydrogen Gas Ratio | 16.4 | 16.7 | 17.1 | 19.3 | 17.1 | 18.6 | 17.0 |
| H2/C2 (ppm/mol %) | | | | | | | |
| Comonomer Gas | 0.0087 | 0.0093 | 0.0099 | 0.0116 | 0.0106 | 0.0110 | 0.0117 |
| Ratio CX/C2 | | | | | | | |
| (mol/mol) | | | | | | | |
| Isopentane | 10.1 | 9.9 | 9.9 | 10.0 | 10.0 | 10.4 | 9.9 |
| Concentration | | | | | | | |
| (mol %) | | | | | | | |

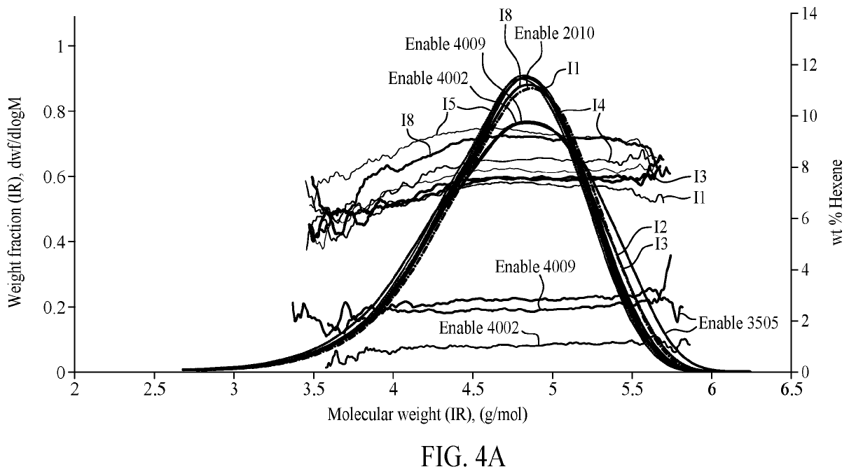

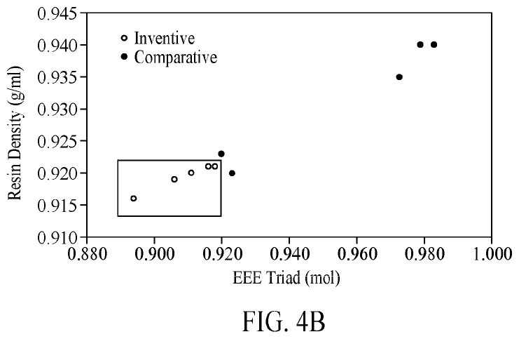

FIG. 4A is a gel permeation chromatography chromatogram of inventive and comparative resins. As shown in FIG. 4A, traces of inventive resins show similar average molecular weight and MWD as commercial resins. All inventive samples show evidence of LCB with a similar level of LCB seen in the value of g′.

FIG. 4B is a graph illustrating density versus EEE triad content of inventive and comparative resins, according to an embodiment. FIG. 4B shows that the inventive resins have a unique combination of density and EEE triad content as compared to the comparative commercial resins.

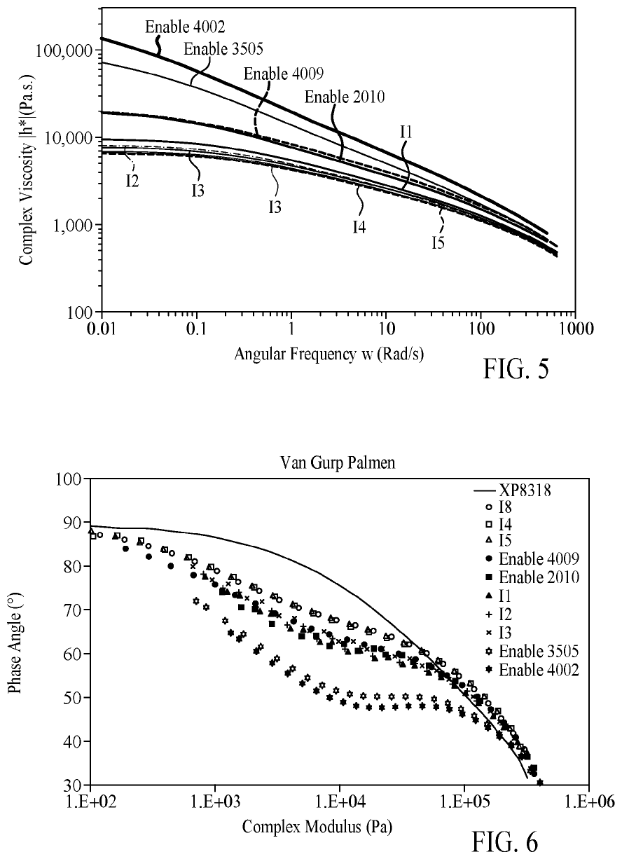

Viscosity flow data using SAOS data are shown in FIG. 5. Viscosities at approximately 100 rad/s (η100) and 0.01 rad/s (η0.01) for the inventive and comparative resins are shown in Table 1. Shear rates close to 100 to 1000 rad/s are typically encountered during the extrusion phase of film fabrication. I8 and C1 (ENABLE™ 2010ME) exhibit similar is viscosities in the typical shear rate range of the extrusion process (approximately 30-300 rad/s) and differ more largely at lower shear rates approaching zero where the starting torque is determined. 18 has a viscosity that is substantially lower than that of C1 and thus would be expected to involve a lower screw torque for starting the extrusion process. Indeed, Table 3 (below) illustrates the overall lower melt pressure and motor load achieved when processing I8 vs C1.

Referring back to FIG. 5, all inventive resins show a significantly lower η_{0.001} compared to the comparative resins. The presence of small amounts of long chain branches (LCB) in ENABLE™ grades is understood to improve processability in cast film applications. A lower viscosity (at low shear rates, 0.01 rad/s) of the inventive resins compared to the commercial grade can lead to lower head pressures and temperatures during extrusion which can lead to increased output. LCB generally results in processing advantages such as reduced neck-in and increased draw stability.

Commercial ENABLE™ grades (C1-C4) contain LCB that not only positively impacts processability but also leads to unique film properties. Hence it was desirable for some embodiments to maintain a critical level of LCB in order not to impact those film properties. FIG. 6 shows VGP (Van Gurp Palmen) curves for inventive and comparative commercial resins. All inventive grades show an inflection point in the curve, which is indicative for the presence of LCB. Exceed™ XP 8318 (LLDPE without such LCB) is included in FIG. 6 as a comparative illustration of a VGP curve for LLDPE without any LCB. Thus, polyethylene copolymers of the present disclosure can be characterized as having an inflection point in a Van Gurp Palmen curve.

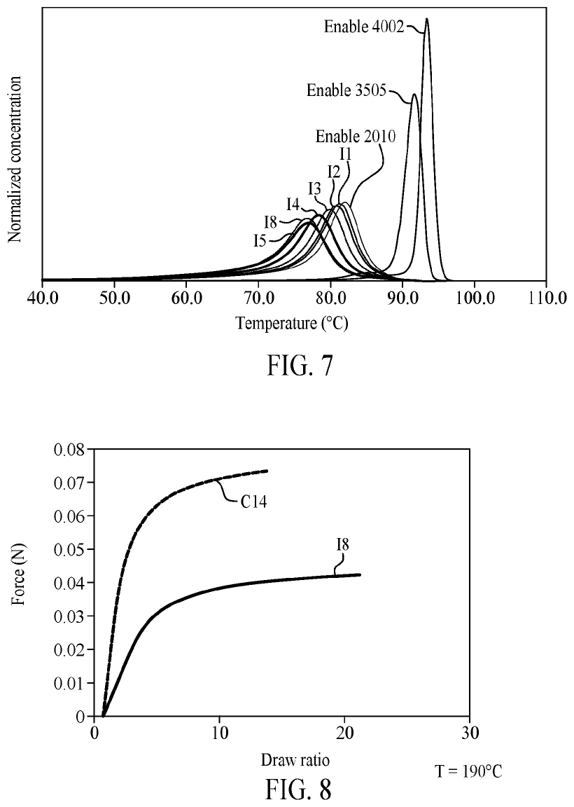

The co-monomer distribution in comparative and inventive grades was determined on the basis of TREF results. FIG. 7 show that with increasing amount of co-monomer in the sample, the peak elution temperature decreases. 18 has the lowest peak temperature compared to C1, C2 and C4. The breadth of the distribution also increases as the amount of co-monomer increases with 18 showing a visible broader chemical composition distribution when compared to C1, C2 and C4.

FIG. 8 is a graph illustrating melt strength of 18 and C14. At 190° C. the melt strength of 18 is approximately half of that of C1.

### Example 2

FIG. 9 shows a radar plot comparing melt pressure, melt temperature and motor load for either using 18 or C1 for the fabrication of a film on a cast line, using the same run conditions for the 18-based cast film and the C1-based cast film. Table 3 shows the corresponding extruder variables for I8 and C1 in connection with this example.

TABLE 3
Extruder parameters for I8 and C1 during Cast film fabrication.

| | I8 | C1 |
|---|---|---|
| Melt pressure (bar) | 190 | 252 |
| Melt temperature (° C.) | 286 | 287 |
| Motor load (%) | 36 | 39 |

### Example 3

FIG. 10 shows a similar radar plot comparing melt pressure, melt temperature and motor load generated by using either 12 or C1 for the fabrication of a blown film on a 5 layer line. Table 4 shows the corresponding extruder variables for 12 and C1 in connection with the Example 3 fabrication data depicted in FIG. 10.

TABLE 4
Extruder parameters for I2 and C1 during Blown film fabrication

| | I2 | C1 |
|---|---|---|
| Melt pressure (bar) | 321 | 363 |
| Melt temperature (° C.) | 204 | 206 |
| Motor load (%) | 45 | 50 |

### Example 4

In a further experiment, blown films were made on a W & H 5 Layer blown film line. In one example, 12 was used as skin layer and an LLDPE having substantially no long chain branching, produced using a pair of metallocene catalysts and having a broad orthogonal composition distribution was used as the core layer (“2-cat LLDPE”). The density of this LLDPE were 0.920 g/cc and the MI were 0.80 g/10 min. This LLDPE did not contain any PPA. Table 5.1 shows the film line parameters; Table 5.2 shows the process parameters at each extruder used in the coextruded blown film production.

TABLE 5.1
Blown Film Parameters for Example 4

| Parameter | Value | |
|---|---|---|
| Die Diameter | 280 | mm |
| Die Gap | 1.4 | mm |
| Target Gauge | 50 | μm |
| BUR (Blow up ratio) | 2.5 | |
| Frost Line Height | 850 | mm |
| Line Speed | 37.2 | m/min |

TABLE 5.2
Example 4 extrusion process parameters

| | | | | 2-cat | | |
|---|---|---|---|---|---|---|
| | | | | LLDPE- | | |
| Resin | | I2 | I2 | 926 | I2 | I2 |
| Extruder | | D | C | B | A | E |
| Output | kg/hr | 28.2 | 28.3 | 112.9 | 28.6 | 27.6 |
| Output | lb/hr/in | 62.2 | 62.4 | 248.9 | 63.1 | 60.9 |
| Melt | bar | 207 | 176 | 314 | 167 | 182 |
| Pressure | psi | 3002 | 2553 | 4554 | 2422 | 2640 |
| Melt | ° C. | 195 | 195 | 225 | 196 | 201 |
| Temperature | ° F. | 383 | 383 | 437 | 385 | 394 |
| Motor Load | % | 36 | 30 | 47 | 40 | 36 |
| Feed Throat | C. | 40 | 40 | 40 | 40 | 40 |
| Temperature | | | | | | |
| Barrel #1 | C. | 180 | 180 | 190 | 180 | 180 |
| Temperature | | | | | | |
| Barrel #2 | C. | 190 | 190 | 200 | 190 | 190 |
| Temperature | | | | | | |
| Barrel #3 | C. | 190 | 190 | 200 | 190 | 190 |
| Temperature | | | | | | |
| Barrel #4 | C. | 190 | 190 | 200 | 190 | 190 |
| Temperature | | | | | | |
| Barrel #5 | C. | 190 | 190 | 200 | 190 | 190 |
| Temperature | | | | | | |
| Zone #6 | C. | 190 | 190 | 200 | 190 | 190 |
| Temperature | | | | | | |
| Die Zone #1 | C. | 200 | 200 | 200 | 200 | 200 |
| Temperature | | | | | | |
| Die Zone #2 | C. | 200 | 200 | 200 | 200 | 200 |
| Temperature | | | | | | |
| Die Zone #3 | C. | 200 | 200 | 200 | 200 | 200 |
| Temperature | | | | | | |
| Die Zone #4 | C. | 200 | 200 | 200 | 200 | 200 |
| Temperature | | | | | | |

In another example, I3 was used in the same blown film line, with the same parameters as in Table 5.1 above (except line speed was 37.1 m/min instead of 37.2 m/min). I3 was used as skin layer and the same 2-cat LLDPE as Example 4 was used as the core layer (“2-cat LLDPE”). The density of this LLDPE were 0.920 g/ml and the MI were 0.80 g/10 min. This LLDPE did not contain any PPA.

Run conditions for the extruders of Example 5 are described in Table 6.

TABLE 6
Example 5 extrusion process parameters

| | | | | 2-cat | | |
|---|---|---|---|---|---|---|
| | | | | LLDPE- | | |
| Resin | | I3 | I3 | 926 | I3 | I3 |
| Extruder | | D | C | B | A | E |
| Output | kg/hr | 27.8 | 28.5 | 112.7 | 28.6 | 28.1 |
| Output | lb/hr/in | 61.3 | 62.8 | 248.5 | 63.1 | 62.0 |
| Melt | bar | 199 | 169 | 310 | 160 | 176 |
| Pressure | psi | 2886 | 2451 | 4496 | 2321 | 2553 |
| Melt | ° C. | 195 | 195 | 225 | 195 | 200 |
| Temperature | ° F. | 383 | 383 | 437 | 383 | 392 |
| Motor Load | % | 34 | 30 | 48 | 39 | 35 |
| Feed Throat | C. | 40 | 40 | 40 | 40 | 40 |
| Temperature | | | | | | |
| Barrel #1 | C. | 180 | 180 | 190 | 180 | 180 |
| Temperature | | | | | | |
| Barrel #2 | C. | 190 | 190 | 200 | 190 | 190 |
| Temperature | | | | | | |
| Barrel #3 | C. | 190 | 190 | 200 | 190 | 190 |
| Temperature | | | | | | |
| Barrel #4 | C. | 190 | 190 | 200 | 190 | 190 |
| Temperature | | | | | | |
| Barrel #5 | C. | 190 | 190 | 200 | 190 | 190 |
| Temperature | | | | | | |
| Zone #6 | C. | 190 | 190 | 200 | 190 | 190 |
| Temperature | | | | | | |
| Die Zone #1 | C. | 200 | 200 | 200 | 200 | 200 |
| Temperature | | | | | | |
| Die Zone #2 | C. | 200 | 200 | 200 | 200 | 200 |
| Temperature | | | | | | |
| Die Zone #3 | C. | 200 | 200 | 200 | 200 | 200 |
| Temperature | | | | | | |
| Die Zone #4 | C. | 200 | 200 | 200 | 200 | 200 |
| Temperature | | | | | | |

### Example 6

Finally, a comparative 5-layer blown film process was carried out, with the same film line parameters as in Table 5.1. A slightly different dual-catalyst LLDPE was used as the core layer (2-cat LLDPE-2), having 0.926 g/cc density and 0.85 g/10 min MI; and the other layers were made using ENABLE™ 2010 (C1), Exceed™ 3812, and/or LD150, as indicated in Table 7 below. Table 7 shows the extruder processing parameters for the 5-layer blown film line in connection with this Example 6.

Although the core was slightly different than Examples 4 and 5, the key comparison here is in the individual extruders processing the commercial resins, as compared to the I2 (Example 4) or I3 (Example 5) resins above, where the superior processability of I2 and I3 compared to the commercial resins can quickly be seen. Moreover, it is important to note that Examples 4 and 5 blown films were made without using polymer processing aid (PPA), and no melt fracture was observed in those films. In contrast, melt fracture is typically seen when resins without processing aid are used. On the other hand, melt fracture is observed when C1 is used.

TABLE 7
Comparative Blown Film Example 6 Extrusion Process Parameters

| | | | | | Exceed | |
|---|---|---|---|---|---|---|
| | | | | | 3812/ | |
| | | | | 2-cat | 40% | Exceed |
| Resin | | Enable 2010 | Enable 2010 | LLDPE-2 | LD150 | 3812 |
| Extruder | | D | C | B | A | E |
| Output | kg/hr | 28.3 | 28.4 | 113 | 28.2 | 27.7 |
| Output | lb/hr/in | 62.4 | 62.6 | 249.2 | 62.2 | 61.1 |
| Melt | bar | 252 | 211 | 322 | 175 | 152 |
| Pressure | psi | 3655 | 3060 | 4670 | 2538 | 2205 |
| Melt | ° C. | 207 | 206 | 222 | 195 | 205 |
| Temperature | ° F. | 405 | 403 | 432 | 383 | 401 |
| Motor Load | % | 36 | 36 | 57 | 40 | 23 |
| Feed Throat | C. | 40 | 40 | 40 | 40 | 40 |
| Temperature | | | | | | |
| Barrel #1 | C. | 190 | 190 | 190 | 180 | 180 |
| Temperature | | | | | | |
| Barrel #2 | C. | 200 | 200 | 200 | 190 | 190 |
| Temperature | | | | | | |
| Barrel #3 | C. | 200 | 200 | 200 | 190 | 190 |
| Temperature | | | | | | |
| Barrel #4 | C. | 200 | 200 | 200 | 190 | 190 |
| Temperature | | | | | | |
| Barrel #5 | C. | 200 | 200 | 200 | 190 | 190 |
| Temperature | | | | | | |
| Zone #6 | C. | 200 | 200 | 200 | 190 | 190 |
| Temperature | | | | | | |
| Die Zone #1 | C. | 200 | 200 | 200 | 200 | 200 |
| Temperature | | | | | | |
| Die Zone #2 | C. | 200 | 200 | 200 | 200 | 200 |
| Temperature | | | | | | |
| Die Zone #3 | C. | 200 | 200 | 200 | 200 | 200 |
| Temperature | | | | | | |
| Die Zone #4 | C. | 200 | 200 | 200 | 200 | 200 |
| Temperature | | | | | | |

¹2-catalyst LLDPE resin (D = 0.926 g/cc, MI = 0.85 g/10 min)
²EXCEED ™ 3812 resin is available from ExxonMobil Chemical Company of Houston, TX.
³LD 150 resin is available from ExxonMobil Chemical Company of Houston, TX.

It was observed that an approximately 20% reduction in melt pressure is obtained with the inventive resin as compared to the comparative resin, providing increased output of film production. In addition, flow to edge improvement observed provides a functional layer that can be used as a skin layer. There was also a 10% reduction in motor load observed with the inventive resin.

Overall, polyethylene polymers, polymerization processes for making such polyethylene polymers, and films made therefrom provide numerous advantages. Polyethylene polymers can be copolymers having a combination of low density, high melt index, maintained long chain branching, and relatively consistent comonomer incorporation across polymer chains of different lengths (high CDBI) while also providing commercially desirable polymerizations and extrusions of the polyethylene copolymers.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

All priority documents are herein fully incorporated by reference for all purposes and for all jurisdictions in which such incorporation is permitted and to the extent such description is consistent with the present disclosure. Further, all documents and references cited herein, including testing procedures, publications, patents, journal articles, etc. are herein fully incorporated by reference for all jurisdictions in which such incorporation is permitted and to the extent such description is consistent with the disclosure.

Likewise whenever a composition, an element or a group of elements is preceded with the transitional phrase “comprising”, it is understood that we also contemplate the same composition or group of elements with transitional phrases “consisting essentially of,” “consisting of”, “selected from the group of consisting of,” or “is” preceding the recitation of the composition, element, or elements and vice versa. The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the claimed invention, additionally, the phrases do not exclude impurities and variances normally associated with the elements and materials used.

While the claimed invention is described with respect to a number of embodiments and examples, those skilled in the art, having benefit of this disclosure, will appreciate that other embodiments can be devised which do not depart from the scope and spirit of the disclosure.

What is claimed is:

1. A polyethylene copolymer, comprising:
ethylene units; and
1 wt % to 8 wt % of C₃-C₈ alpha-olefin comonomer units, the polyethylene copolymer having:
a density of 0.914 g/cm³ to 0.925 g/cm³,
a melt index (MI, determined per ASTM D1238 at 190° C. and 2.16 kg loading) greater than 1 g/10 min and less than or equal to 2.5 g/10 min,
a composition distribution breadth index of 75% or greater, and
a molecular weight distribution (M_{w}/Mₙ) of 2 to 8.

2. The polyethylene copolymer of claim 1, having long chain branching.

3. The polyethylene copolymer of claim 1, having one or more of the following:
(i) a melt index ratio (MIR) within the range from 25 to 40, wherein MIR is the ratio of high load melt index (HLMI, ASTM D1238 at 190° C., 21.6 kg) to melt index (MI, ASTM D1238 at 190° C., 2.16 kg);
(ii) an inflection point in a Van Gurp Palmen plot of phase angle vs. complex modulus (Pa) of the polyethylene copolymer; and
(iii) a shear thinning ratio less than 15.

4. The polyethylene copolymer of claim 1, wherein the polyethylene copolymer has a Q parameter of 50 to 90, as determined by Equation (1):
Q parameter=0.001*(MIR*η_{0.001}/η₁₀₀)²*(T₇₅−T₂₅)/MWD (Eq. 1)
wherein MIR is melt index ratio, η_{0.001} is complex shear viscosity (η*)@0.01 rad/sec and 190° C., η₁₀₀ is complex shear viscosity (η*)@100 rad/sec and 190° C., T₇₅−T₂₅ is T₇₅−T₂₅ value, wherein T₂₅ is the temperature at which 25% of eluted polyethylene copolymer is obtained and T₇₅ is the temperature at which 75% of eluted polyethylene copolymer is obtained using temperature rising elution fractionation, and MWD is molecular weight distribution.

5. The polyethylene copolymer of claim 1, wherein the polyethylene copolymer has 0.1 to 0.15 trisubstituted vinylenes/1000 carbon atoms.

6. The polyethylene copolymer of claim 1, wherein the polyethylene copolymer has an [EEE]triad content of 89 mol % to 91.8 mol %.

7. The polyethylene copolymer of claim 1, wherein the polyethylene copolymer has:
an [HEE]triad content of 3 mol % to 8 mol %,
an [HEH]triad content of 0.05 mol % to 0.5 mol %,
an [EHE]triad content of 1 mol % to 5 mol %,
an [EHH]triad content of 0.05 mol % to 0.5 mol %, and
an [HHH]triad content of 0.05 mol % to 0.5 mol %.

8. The polyethylene copolymer of claim 1, wherein the polyethylene copolymer has a density of 0.918 g/cm³ to 0.922 g/cm³.

9. The polyethylene copolymer of claim 1, wherein the polyethylene copolymer has a density of 0.915 g/cm³ to 0.920 g/cm³.

10. The polyethylene copolymer of claim 1, wherein the C₃-C₈ alpha-olefin comonomer units are 1-hexene units and the polyethylene copolymer comprises 92 wt % to 99 wt % ethylene.

11. The polyethylene copolymer of claim 1, wherein the polyethylene copolymer further has one or more of the following properties:
(a) MI of 1.5 g/10 min to 2.3 g/10 min;
(b) a T₇₅−T₂₅ value of 4° C. to 10° C.;
(c) a high load melt index (HLMI, determined per ASTM D1238 at 190° C. and 21.6 kg loading) of 45 g/10 min to 70 g/10 min;
(d) a melt index ratio (MIR, HLMI/MI) of 27 to 33;
(e) a weight-average molecular weight (Mw) of 70,000 g/mol to 95,000 g/mol; or
(f) a number-average molecular weight (Mn) of 20,000 g/mol to 40,000 g/mol.

12. The polyethylene copolymer of claim 11, having all of the properties (a)-(f).

13. The polyethylene copolymer of claim 1, wherein the polyethylene copolymer has an HLMI of 45 g/10 min to 55 g/10 min.

14. The polyethylene copolymer of claim 1, wherein the polyethylene copolymer has an HLMI of 60 g/10 min to 70 g/10 min.

15. The polyethylene copolymer of claim 1, wherein the polyethylene copolymer has:
a complex shear viscosity (η*)@0.01 rad/sec and 190° C. of 6,000 Pa·s to 8,000 Pa·s, and a complex shear viscosity (η*)@100 rad/sec and 190° C. of 1,100 Pa·s to 1,300 Pa·s.

16. A cast film comprising the polyethylene copolymer of claim 1, wherein the polyethylene copolymer has a density of 0.915 g/cm³ to 0.917 g/cm³ and a melt index of 1.6 g/10 min to 1.8 g/10 min.

17. A blown film comprising the polyethylene copolymer of claim 1, wherein the polyethylene copolymer has a density of 0.918 g/cm³ to 0.922 g/cm³ and a melt index of 1.5 g/10 min to 2.3 g/10 min.

18. The blown film of claim 17, wherein the blown film is substantially free of a per-fluoro alkane or a poly-fluoro alkane.

19. A method comprising:
measuring a stickiness temperature of a polyethylene copolymer at each of a plurality of concentrations of an induced condensing agent in a testing device;
measuring a density, a melt index (MI), and a high load melt index (HLMI) of the polyethylene copolymer;
calculating a melt flow ratio by dividing the HLMI by the MI;
calculating an equivalent partial pressure of the induced condensing agent, the equivalent partial pressure corresponding to a partial pressure of isomers accumulating in a reactor; and
determining an equation that relates the stickiness temperature to the equivalent partial pressure, based, at least in part, on the density, the MI, and the MFR of the polyethylene copolymer, and
using the equation, producing an additional amount of the polyethylene copolymer,
wherein the polyethylene copolymer comprises:
ethylene units; and
1 wt % to 8 wt % of C₃-C₈ alpha-olefin comonomer units, the polyethylene copolymer having:
a density of 0.914 g/cm³ to 0.925 g/cm³,
a melt index (MI, determined per ASTM D1238 at 190° C. and 2.16 kg loading) greater than 1 g/10 min and less than or equal to 2.5 g/10 min,
a composition distribution breadth index of 75% or greater, and
a molecular weight distribution (M_{w}/Mₙ) of 2 to 8.

20. A method of making the polyethylene copolymer of claim 1, the method comprising:
measuring one or more parameters for a polymerization reaction, said one or more parameters including a reactor temperature and a concentration of an induced condensing agent (ICA) in a polymerization reactor;
calculating an equivalent partial pressure of the ICA;
locating the polymerization reaction in a two-dimensional space on a plot defined by a reactor temperature dimension and an equivalent partial pressure dimension;
comparing the location of the polymerization reaction in the two-dimensional space to a non-sticking regime, the non-sticking regime defined as a space between an upper temperature limit curve and a lower temperature limit curve;
adjusting the one or more parameters of the polymerization reaction to operate the polymerization reaction within the non-sticking regime; and
based on the adjusted one or more parameters, obtaining the polyethylene copolymer of any one of claims 1 to 15 from the polymerization reactor.
